(19) United States
(12) Patent Application Publication
(10) Pub. No.: US 2017/0067012 A1
(43) Pub. Date: Mar. 9, 2017

(54) HYALURONIC ACID DERIVATIVES AND COMPOSITION FOR CELL-SURFACE ENGINEERING USING THE SAME

(71) Applicant: POSTECH ACADEMY-INDUSTRY FOUNDATION, Pohang-si (KR)

(72) Inventors: Sei Kwang HAHN, Pohang-si (KR); Yun seop KIM, Seoul (KR); Won Ho KONG, Pohang-si (KR); Hyemin KIM, Daegu (KR)

(21) Appl. No.: 15/249,285

(22) Filed: Aug. 26, 2016

(30) Foreign Application Priority Data
Aug. 27, 2015 (KR) 10-2015-0120849
Aug. 24, 2016 (KR) 10-2016-0107904

(51) Int. Cl. C12N 5/00; C08B 37/08

(52) U.S. Cl. CPC C12N 5/0006; C08B 37/0072

(57) ABSTRACT

Provided are a hyaluronic acid derivative for liver-targeting delivery via intravenous injection of cells and a preparation method thereof. More particularly, provided are a preparation method of a hyaluronic acid derivative capable of modifying the surface of cells and also having biocompatibility, biodegradability, and liver-targeting deliver property, and use of the hyaluronic acid derivative prepared thereby as a liver-targeting cell delivery system.

### TECHNICAL FIELD

The present disclosure relates to a hyaluronic acid derivative, a method of preparing the same, and a cell engineering composition for the treatment of diseases including the hyaluronic acid derivative, and a liver-targeting cell delivery system.

### BACKGROUND

A cell delivery system is a system that delivers cells themselves and a drug of a genetically engineered cell delivery system via a variety of injection routes such as intra-arterial injection, intravenous injection, subcutaneous injection, topical injection, intra-peritoneal injection, etc., and treatment of various diseases and tissue regeneration are possible by using the cell delivery system.

A variety of cells such as stem cells, immune cells, etc. may be used as a cellular therapeutic agent. In particular, mesenchymal stem cells have advantages of inducing a relatively weak immune response, having immunomodulatory ability, and differentiating into many different types of cells, and therefore, they are commonly applied to cell carriers for drug delivery, disease treatment, and tissue regeneration.

In this regard, cell delivery to a target site is one of important factors that should be considered in many cell delivery systems including mesenchymal stem cells. Currently, many methods are used for delivery of cells to a target site, but topical injection and intra-arterial injection, compared to intravenous injection, may cause the risk and side effects such as bleeding and tissue damages. Therefore, delivery of cells to a target site via intravenous injection is considered as a simple ideal method.

At present, delivery of immune cells or stem cells to the liver via intravenous injection and hepatic portal venous injection/hepatic arterial injection is being studied for the treatment of liver diseases such as liver cancer or cirrhosis. However, cells must be cultured in-vitro for a predetermined period of time to provide cells with cancer targeting specificity, and intravenous injection of cells results in the entrapment of a number of cells mainly in lungs, causing low delivery efficiency of cells to the liver. Further, hepatic portal venous injection/hepatic arterial injection requires laparotomy. Accordingly, there is a demand for the development of a liver-targeting delivery system, which enables efficient delivery of cells to the liver via simple intravenous injection, thereby being applied to the treatment of all liver diseases.

### DISCLOSURE

### Technical Problem

An object of the present invention is to provide a hyaluronic acid derivative including units represented by the following Chemical Formulae 1 to 3 or a salt thereof; or a hyaluronic acid derivative including units represented by the following Chemical Formulae 1 and 4 or a salt thereof:

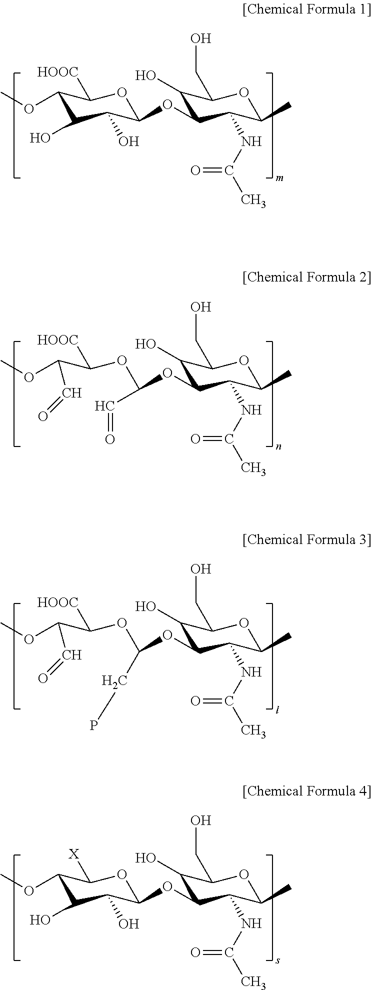

wherein P is a water-soluble protein;

X is —COR1, —CONR2H—R1 or —CONH—R2—NH—R1;

R1 is a vinyl group, a thiol group, poly-L-lysine, a lipid, an acrylate group, a maleimide group, or N-hydroxysuccinimide (NHS);

R2 is an alkyl group having 1 to 6 carbon atoms; and

the sum of m, n, and 1 is an integer of 50 to 10,000, the sum of n and 1 is an integer of 5 to 5,000, 1 is an integer of 1 to 100, and s is an integer of 1 to 2000.

Another object of the present invention is to provide a method of preparing the hyaluronic acid derivative or the salt thereof.

Still another object of the present invention is to provide a cell surface modification composition including the hyaluronic acid derivative or the salt thereof.

Still another object of the present invention is to provide cells which are surface-modified with the hyaluronic acid derivative or the salt thereof, or a pharmaceutical composition including the same as an active ingredient.

Still another object of the present invention is to provide a method of treating a liver disease by administering the pharmaceutical composition including the cells which are surface-modified with the hyaluronic acid derivative or the salt thereof as an active ingredient.

Still another object of the present invention is to provide a method of targeting the liver with active cells using the hyaluronic acid derivative, the method including the step of administering the hyaluronic acid derivative to a subject in need thereof.

### Technical Solution

The experimental results of the present inventors demonstrated that hyaluronic acid, a biopolymer having superior biodegradability and biocompatibility, has liver-targeting properties, and therefore, when hyaluronic acid having excellent liver-targeting capability is applied to cell surface modification for target delivery of cells to the liver, delivery of the cells not to the lung but to the liver is possible. By using these properties, a cell modification composition including a hyaluronic acid derivative for the treatment of diseases and a preparation method thereof were developed, and they may be effectively applied for the treatment of liver diseases via intravenous injection of a cell therapeutic agent, thereby completing the present invention.

Cells surface-modified with the hyaluronic acid derivative according to the present invention may be specifically delivered to the liver via intravenous injection. More particularly, the present disclosure relates to preparation of the hyaluronic acid derivative for cell surface modification and a cell therapy system for liver-targeting delivery via intravenous injection of cells surface-modified with the hyaluronic acid derivative prepared thereby, in which the hyaluronic acid derivative capable of efficiently modifying cell surface and also having biocompatibility, biodegradability, and liver-targeting properties may be safely applied to the human body, and may be applied to a target delivery system of various cells to the liver via intravenous injection, thereby being utilized in a drug delivery system of a cell carrier and tissue engineering.

An aspect of the present invention provides a cell surface modification composition, including a hyaluronic acid derivative including units represented by the following Chemical Formulae 1 to 3 or a salt thereof; or a hyaluronic acid derivative including units represented by the following Chemical Formulae 1 and 4 or a salt thereof:

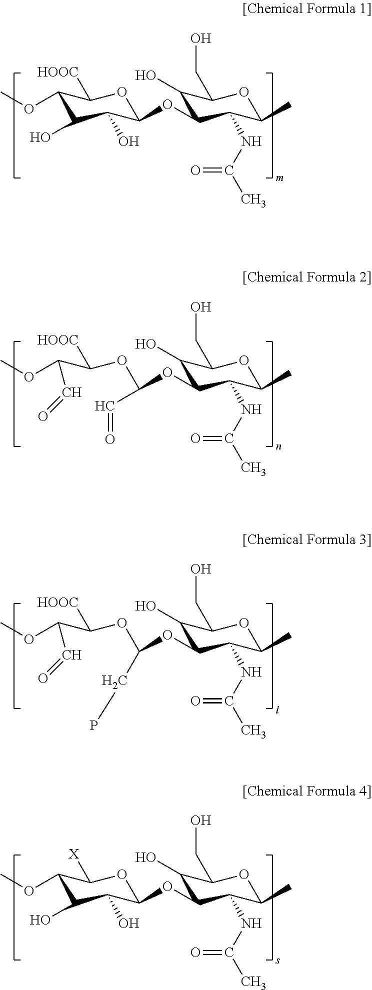

wherein P is a water-soluble protein;

X is —COR¹, —CONR²H—R¹ or —CONH—R²—NH—R¹;

R¹ is a vinyl group, a thiol group, poly-L-lysine, a lipid, an acrylate group, a maleimide group, or N-hydroxysuccinimide (NHS);

R² is an alkyl group having 1 to 6 carbon atoms; and

the sum of m, n, and 1 is an integer of 50 to 10,000, the sum of n and 1 is an integer of 5 to 5,000, 1 is an integer of 1 to 100, and s is an integer of 1 to 2000.

Another aspect of the present invention provides a cell surface modification composition, including a hyaluronic acid derivative represented by the following Chemical Formula 5 or a salt thereof:

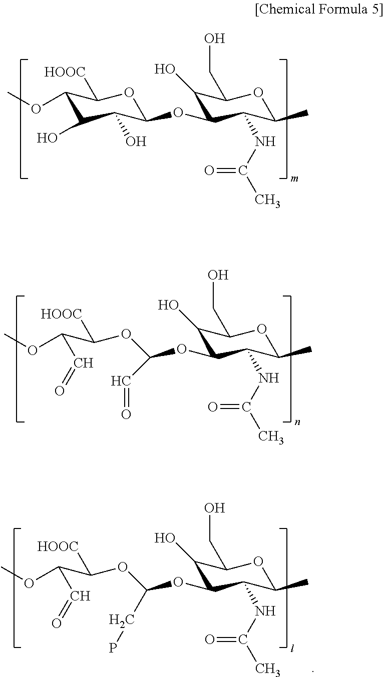

wherein P is a water-soluble protein; the sum of m, n, and 1 is an integer of 50 to 10,000, the sum of n and 1 is an integer of 5 to 5,000, and 1 is an integer of 1 to 100.

Preferably, the water-soluble protein may be a wheat germ agglutinin (hereinafter, referred to as WGA) protein. A specific example of WGA may be a protein having an amino acid sequence of SEQ ID NO: 1.

The water-soluble protein bound to the hyaluronic acid derivative is characterized by maintaining a secondary structure of the water-soluble protein before binding, even though it binds to hyaluronic acid.

The water-soluble protein is conjugated to hyaluronic acid to facilitate interaction with N-acetyl glucosamine and/or sialic acid which is one of glycoproteins present on the cell surface, thereby effectively modifying the cell surface. Specifically, the conjugation may occur by chemical bonding of the N-terminal amine group of the water-soluble protein and aldehyde of the hyaluronic acid derivative.

In particular, the conjugation may be performed under a particular pH condition to prevent a reaction with other amino acids having another amine groups in the proteins, such as lysine, etc., and therefore, activity of the protein conjugated to hyaluronic acid may be maximized to maximize cell surface modification.

Still another aspect of the present invention provides a cell surface modification composition, including a hyaluronic acid derivative represented by the following Chemical Formula 6 or a salt thereof:

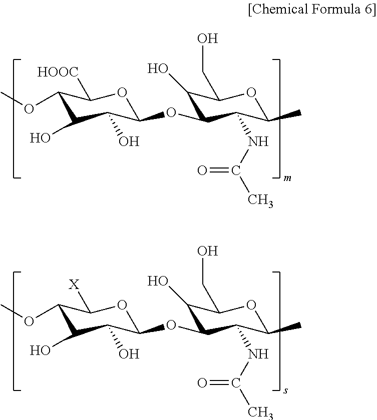

X is —COR¹, —CONR²H—R¹ or —CONH—R²—NH—R¹; R¹ is a vinyl group, a thiol group, a poly-L-lysine, a lipid, an acrylate group, a maleimide group, or N-hydroxysuccinimide (NHS); R² is an alkyl group having 1 to 6 carbon atoms; the sum of m and s is an integer of 50 to 10,000, and s is an integer of 1 to 2000.

Preferably, in Chemical Formula 6, X is —COR¹, R¹ is a lipid, and the sum of m and s is an integer of 50 to 10,000.

Preferably, in Chemical Formula 6, X is —COR¹, R¹ is a thiol group, and the sum of m and s is an integer of 50 to 10,000.

Preferably, in Chemical Formula 6, X is —CONH—R²—NH—R¹, R¹ is a maleimide group, R² is a hexyl group, and the sum of m and s is an integer of 50 to 10,000.

When X of the hyaluronic acid derivative is a lipid, the hyaluronic acid derivative may bind to a cell in the form of inserting a new lipid into the cell membrane composed of a lipid bilayer, and when X is NHS, acrylate, thiol, or maleimide, the hyaluronic acid derivative may bind to a cell by forming a covalent bond with an amine group or a thiol group of a protein present on the cell surface.

Preferably, when surface modification is performed using maleimide-PEG and maleimide-PEG-lipid in this order with a time interval, in addition to the hyaluronic acid-thiol derivative, a click reaction between the hyaluronic acid-thiol derivative and maleimide occurs to form a conjugate, in which maleimide, maleimide-PEG, or maleimide-PEG-lipid is chemically bound to the bound hyaluronic acid derivative. As a result, a thick cell modification face is maintained to easily control an influx rate of the hyaluronic acid derivative into cells.

There is no limitation in the constitution of the hyaluronic acid, but the hyaluronic acid may be preferably hyaluronic acid having a molecular weight of 100,000 to 3,000,000 Da, and more preferably 100,000 to 1,000,000 Da in order to optimize its use in the hyaluronic acid derivative or the salt thereof for cell surface modification and liver-targeting cell delivery.

Meanwhile, the term “hyaluronic acid (HA)”, as used herein, refers to a polymer having a repeating unit represented by the following Chemical Formula 7, and includes all salts of hyaluronic acid, unless the context clearly indicates otherwise.

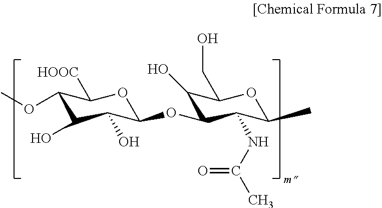

wherein m″ may be an integer of 50 to 10,000.

Still another aspect provides a method of preparing a hyaluronic acid derivative including units represented by the following Chemical Formula 1 to 3 or a salt thereof, the method including a first step of preparing hyaluronic acid-aldehyde (HA-aldehyde) represented by the following Chemical Formula 8 by reacting hyaluronic acid with an oxidant in the dark condition to open the ring structure of the hyaluronic acid; and a second step of reacting the hyaluronic acid-aldehyde and the N-terminus of a water-soluble protein in an aqueous solution:

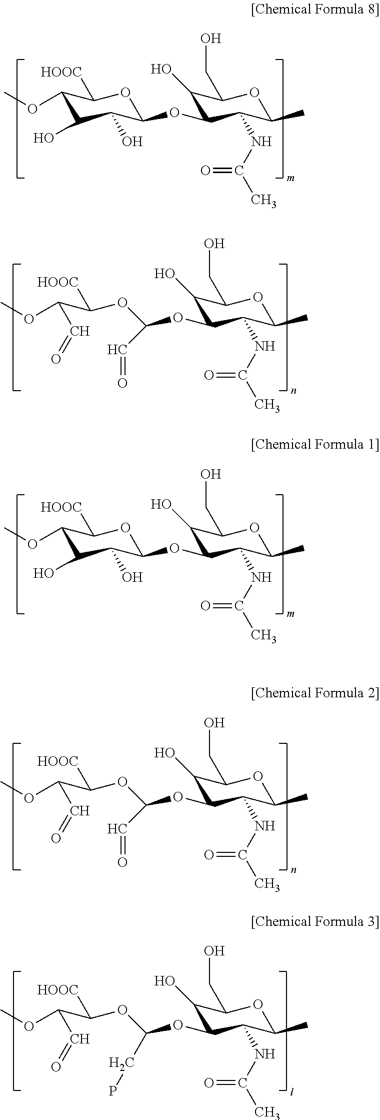

wherein the sum of m and n is an integer of 50 to 10,000 and n is an integer of 5 to 5,000;

P is a water-soluble protein; and

the sum of m, n and 1 is an integer of 50 to 10,000, the sum of n and 1 is an integer of 5 to 5,000, and 1 is an integer of 1 to 100.

The above descriptions of the hyaluronic acid derivatives or salts thereof may be also applied to the method of preparing the hyaluronic acid derivative or the salt thereof.

According to the preparation method of the present invention, the surface of the cells to be modified and the derivative region in the hyaluronic acid derivative, namely, a WGA protein, a thiol group, a lipid, NHS, acrylate, thiol, or maleimide are reacted with each other to modify the cell surface. In particular, when a hyaluronic acid derivative including a carboxyl group is used to modify the surface of cells, and then the cells are injected intravenously, CD44 present in the liver or HARE (hyaluronic acid receptor for endocytosis) abundant in liver sinusoidal endothelial cells binds with the carboxyl group, thereby maximizing the liver-targeting delivery.

Preferably, the derivative may include 1 to 10 molecules, and more preferably 2 to 6 molecules of the water-soluble protein per 1 molecule of the hyaluronic acid derivative.

In the hyaluronic acid derivative according to the present invention, a substitution ratio of hyaluronic acid-aldehyde, which is defined as a mole percentage of the repeating unit including an aldehyde group formed by ring opening of the hyaluronic acid relative to the entire repeating unit in the hyaluronic acid derivative, may be expressed as a value greater than 0 but 1 or less, or a value greater than 0 mole % but 100 mole % or less. Preferably, the substitution ratio of hyaluronic acid-aldehyde after the first step may be 10 to 50 mole %, and more preferably 10 to 40 mole %.

When the substitution ratio is within the above range, the amount of the water-soluble protein including WGA which may be involved in the binding becomes sufficient, and therefore, cell surface modification may occur efficiently, and sufficient interaction with hyaluronic acid receptors present in the liver may occur at the same time, thereby obtaining desired targeting effects.

As described above, the biocompatible and biodegradable polymer, hyaluronic acid is bound to the water-soluble protein, which may be safely applied to the human body. In addition, the water-soluble protein is conjugated by opening of the ring structure while leaving the carboxyl group binding to the hyaluronic acid receptor in cells, thereby maximizing the liver tissue-specific delivery property of hyaluronic acid. Therefore, the hyaluronic acid derivative may have various applications in the development of a cell surface modifier as well as a cellular therapeutic agent for the treatment of liver diseases.

The oxidant is not particularly limited, as long as it may open the ring structure of hyaluronic acid, and sodium periodate (NaIO4) may be preferably used.

On the basis of unit of hyaluronic acid, the oxidant may be preferably added in an amount of 1 to 10 mole times, and more preferably, 1 to 5 mole times. Preferably, the oxidant may be reacted for 2 to 24 hours, and preferably, for 2 to 12 hours. When the oxidant was added at 1 mole time and reacted for 4 hours, 10 mole % was substituted. When the oxidant was added at 5 mole times and reacted for 12 hours, the derivative having 40 mole % of substitution may be obtained.

After the reaction of oxidant, a process of purifying the product by dialysis against distilled water may be further performed, preferably.

Preferably, the second step may be performed in a buffer solution. Preferably, the hyaluronic acid-aldehyde is dissolved in the buffer solution, and then a water-soluble protein, for example, WGA is added to react the aldehyde group included in hyaluronic acid-aldehyde with the N-terminal amine group of the water-soluble protein. Since the amine group exists in the N-terminal of the water-soluble protein, for example, WGA, the water-soluble protein used in the second step may be used as it is without an additional preparation process. Further, since the hyaluronic acid-aldehyde and the water-soluble protein are water-soluble, they may be dissolved in water, and then reacted to bind to each other in the form of the aqueous solution.

The buffer solution may be at pH 5 to 7, and more preferably, at pH 5 to 6.5. For example, the buffer solution may be sodium acetate buffer at pH 5 to 6.5. The conjugation process is performed under the pH conditions within the above range, thereby preventing a reaction between hyaluronic acid-aldehyde and other amino acids having another amine groups in the proteins, such as lysine, etc., and therefore, activity of the water-soluble protein, in particular, WGA may be maximized to improve bioconjugation efficiency and cell surface modification efficiency.

Preferably, 1 to 10 molecules, and more preferably 2 to 6 molecules of the water-soluble protein may be reacted with and bound to 1 molecule of hyaluronic acid-aldehyde.

In the second step, the water-soluble protein may be reacted with the addition of sodium cyanoborohydride (NaCNBH₃). Sodium cyanoborohydride function to reduce the double bond occurred after the reaction of the N-terminal amine group of the water-soluble protein with the aldehyde group of the hyaluronic acid derivative. Preferably, sodium cyanoborohydride may be added in an amount of 2 to 10 mole times, preferably, 2 to 5 mole times of the aldehyde group, depending on the substitution ratio of hyaluronic acid-aldehyde, for the reaction of 12 to 24 hours, preferably 12 to 18 hours.

Meanwhile, because the aldehyde group has a property of high reactivity, a third step may be further performed by blocking the remaining aldehyde group unreacted with the protein using amines, after the second step. A substance used for the blocking is not particularly limited, as long as it is able to react with the carboxyl group or aldehyde group. Preferably, the substance may be amines More preferably, the amines may be straight or branched alkyl carbazate having 1 to 5 carbon atoms, or lower alkanolamine having 1 to 5 carbon atoms. Such blocking process may be represented by the following Reaction Scheme 1:

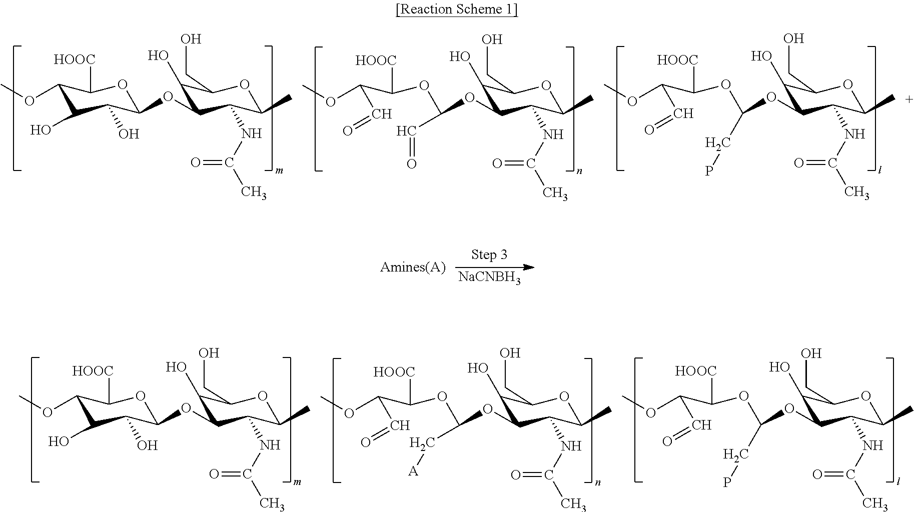

wherein P may be a water-soluble protein, and more preferably, WGA and A is a blocking material.

The blocking material A may be straight or branched alkyl carbazate having 1 to 5 carbon atoms, or lower alkanolamine having 1 to 5 carbon atoms, and the alkyl carbazate may be preferably ethyl carbazate or tert-butyl carbazate, and the alkanolamine may be aminomethanol, aminoethanol, aminopropanol, aminobutanol, or aminopentanol.

Preferably, the amines may be added in an amount of 5 to 10 mole times, preferably 5 to 7 mole times with respect to unit mole of the aldehyde group, and reacted for 12 to 24 hours, preferably for 12 to 18 hours. 100% blocking is possible by adding the amines within the above range.

Preferably, the third step may include a reaction of the following Reaction Scheme 2:

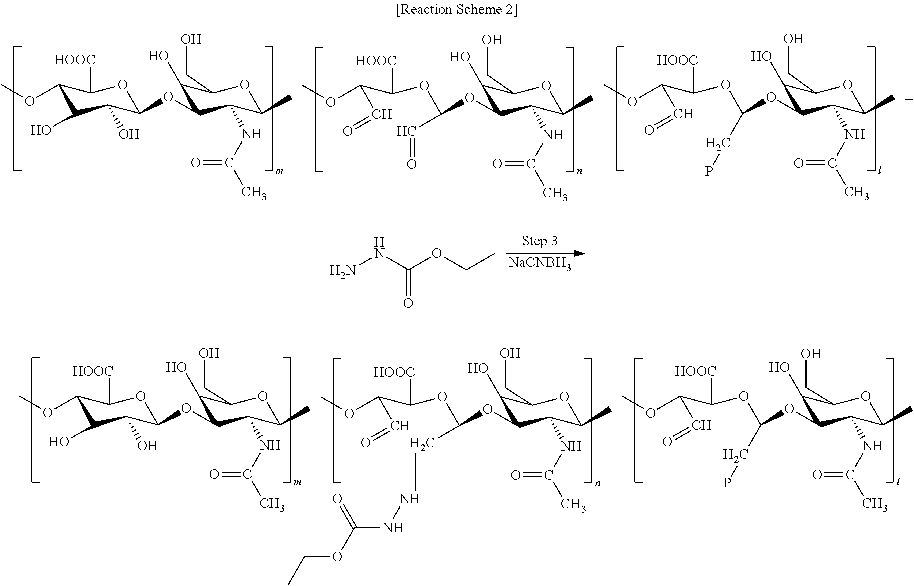

wherein P may be a water-soluble protein, and more preferably, WGA.

More preferably, the method of preparing the hyaluronic acid derivative may include a reaction of the following Reaction Scheme 3:

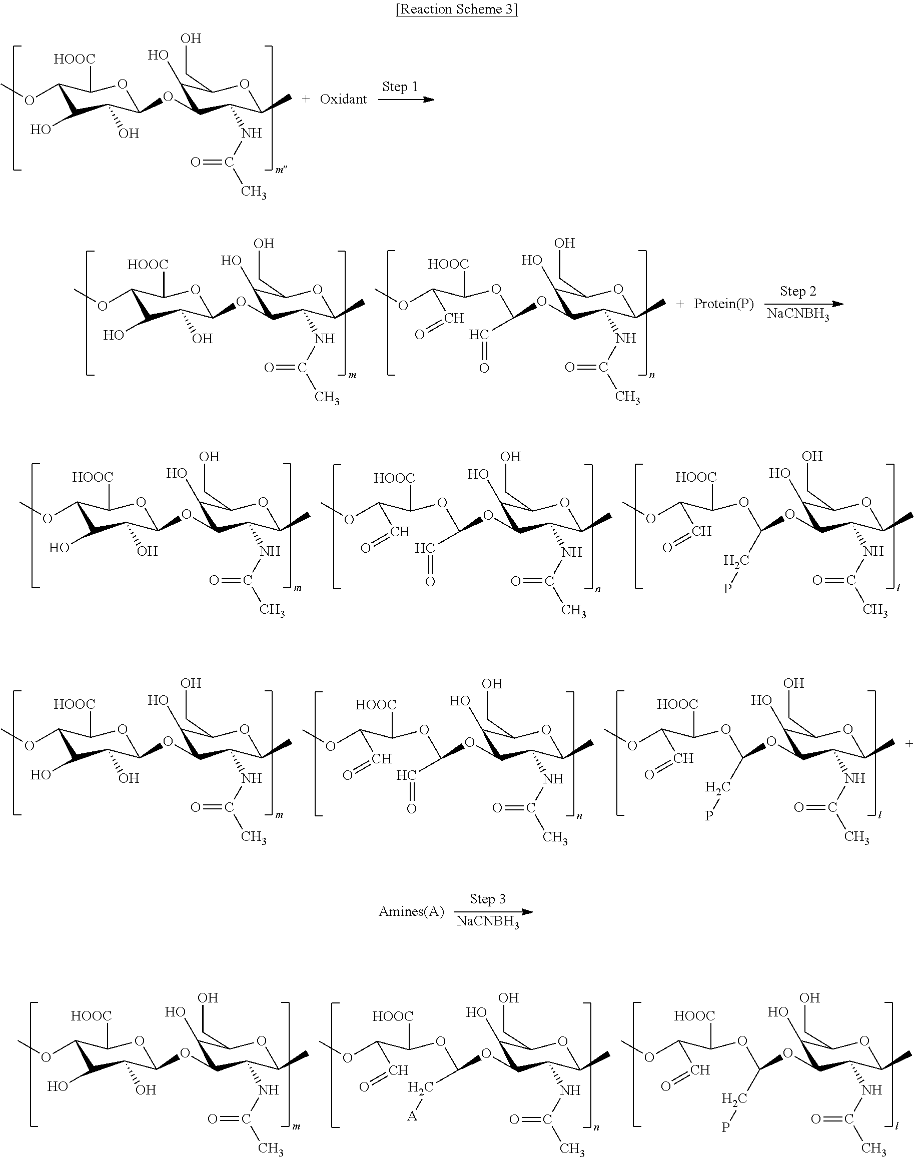

Meanwhile, when alkyl carbazate is used as the amines, the reaction may be preferably allowed under condition of pH 5 to 7. To this end, the acetate buffer as used in the first step may be preferably used. When alkanolamine is used, the reaction may be preferably allowed under condition of pH 7 to 9. To this end, phosphate buffer may be preferably used.

The blocking of the remaining aldehyde groups which do not participate in the reaction may prevent the aldehyde group from reacting with another amine group of WGA bound to the hyaluronic acid derivative. Further, an undesired reaction occurring after the hyaluronic acid derivative according to an embodiment of the present invention enters the body, for example, non-specific reaction of the aldehyde group with various proteins in the body, may be effectively prevented, thereby further improving the efficiency of the desired reaction.

Still another aspect of the present invention provides a liver-targeting cell delivery composition, including a hyaluronic acid derivative including units represented by the following Chemical Formulae 1 to 3 or a salt thereof; or a hyaluronic acid derivative including units represented by the following Chemical Formulae 1 and 4 or a salt thereof:

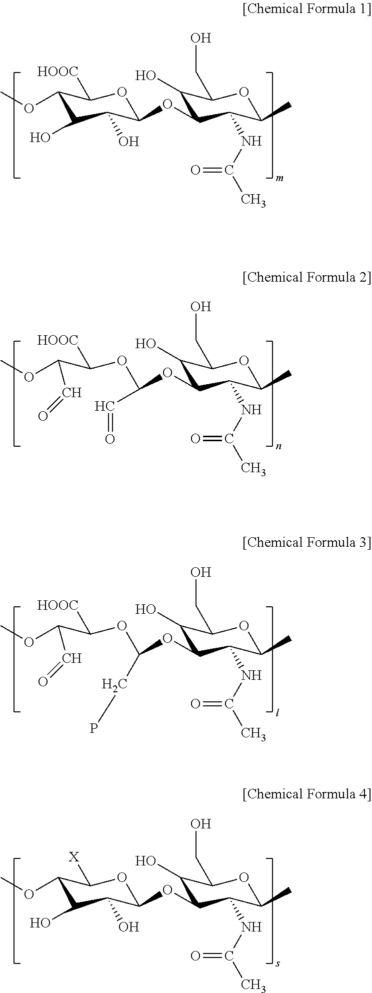

wherein P is a water-soluble protein;

X is —COR¹, —CONR²H—R¹ or —CONH—R²—NH—R¹;

R¹ is a vinyl group, a thiol group, poly-L-lysine, a lipid, an acrylate group, a maleimide group, or N-hydroxysuccinimide (NHS);

R² is an alkyl group having 1 to 6 carbon atoms; and

the sum of m, n, and 1 is an integer of 50 to 10,000, the sum of n and 1 is an integer of 5 to 5,000, 1 is an integer of 1 to 100, and s is an integer of 1 to 2000.

Still another aspect of the present invention provides a liver-targeting cell delivery composition or a liver-targeting cell delivery system, including a hyaluronic acid derivative represented by the following Chemical Formula 5 or Chemical Formula 6 or a salt thereof:

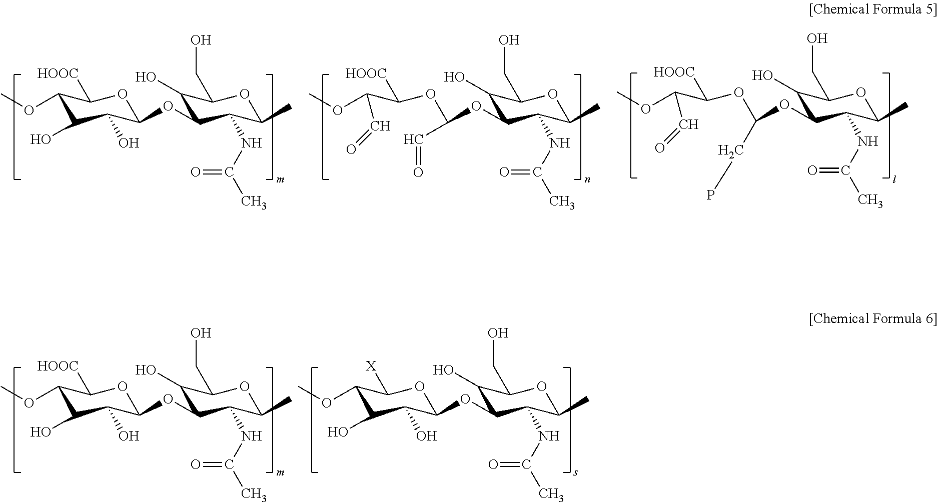

in Chemical Formula 5, P is a water-soluble protein; and

the sum of m, n, and 1 is an integer of 50 to 10,000, the sum of n and 1 is an integer of 5 to 5,000, 1 is an integer of 1 to 100;

in Chemical Formula 6, X is —COR¹, —CONR²H—R¹ or —CONH—R²—NH—R¹;

R¹ is a vinyl group, a thiol group, poly-L-lysine, a lipid, an acrylate group, a maleimide group, or N-hydroxysuccinimide (NHS);

R² is an alkyl group having 1 to 6 carbon atoms; and

the sum of m and s is an integer of 50 to 10,000, and s is an integer of 1 to 2000.

The above descriptions of the hyaluronic acid derivatives or salts thereof may be also applied to the liver-targeting cell delivery composition or the liver-targeting cell delivery system including the hyaluronic acid derivative or the salt thereof.

Further, the present invention provides a cell which is surface-modified with the hyaluronic acid derivative or the salt thereof, or a pharmaceutical composition including the same as an active ingredient.

The above descriptions of the hyaluronic acid derivatives or salts thereof may be also applied to the cell which is surface-modified with the hyaluronic acid derivative or the salt thereof, or the pharmaceutical composition including the same as an active ingredient.

The cells surface-modified with the hyaluronic acid derivative according to an embodiment of the present invention may be cells needed to be delivered to the liver tissue, for example, cellular therapeutic agents for the prevention or treatment of liver diseases or stem cells for liver tissue regeneration. A biopolymer, hyaluronic acid derivative or salt thereof, is biodegradable and biocompatible, and also excellent in the liver tissue-specific delivery property, and therefore, it may be used as a cell surface modifier to deliver the cells not to the lung but to the liver.

The cells may be, are not particularly limited to, cells used as cellular therapeutic agents or stem cells for tissue regeneration, and preferably, mammalian cells, mammalian cell-derived cells, or genetically engineered cells thereof, for example, erythrocytes or stem cells, preferably, mesenchymal stem cells or genetically engineered cells thereof.

Therefore, the cells surface-modified with the hyaluronic acid derivative or the salt thereof, or the pharmaceutical composition including the same as an active ingredient may be used of the prevention or treatment of diseases, depending on the use of the surface-modified cells. Preferably, the diseases may be one or more selected from the group consisting of liver cancer, metastatic liver cancer, hepatic cirrhosis, hepatitis, and hepatic fibrosis, but are not limited thereto. Specific diseases may be determined depending on the cells to be surface-modified with the hyaluronic acid derivative or salt thereof.

The pharmaceutical composition may be administered by one or methods selected from the group consisting of intra-arterial injection, intravenous injection, hepatic portal venous injection, hepatic arterial injection, subcutaneous injection, topical injection, intra-peritoneal injection, oral administration, intranasal administration, and rectal administration, preferably parenteral administration, and more preferably, intravenous injection. When the intravenous injection is used as an administration method, side effects such as bleeding and tissue damages caused by topical injection and intra-arterial injection may be prevented, and laparotomy required for hepatic portal venous injection or hepatic arterial injection may be prevented, and therefore, the administration may be easier. Further, when the known cellular therapeutic agents for liver diseases are injected intravenously, there is a problem that a large number of cells are trapped in the lung, and thus delivery of cells to the liver does not efficiently occur. In contrast, the composition of the present invention may efficiently deliver cellular therapeutic agents to the liver even by intravenous injection, and excellent targeting property and therapeutic effects may be obtained even by a simple method.

The composition of the present invention may further include a filler, an excipient, a disintegrating agent, a binder, a lubricant, etc. Further, the composition of the present invention may be formulated by a method known in the art, such that it may provide rapid, sustained, or delayed release of the active ingredient, after administered to a mammal. The formulation may be in the form of powders, granules, tablets, emulsions, syrups, aerosols, soft or hard gelatin capsules, sterile injectable solutions, or sterile powders, etc.

The pharmaceutical composition of the present invention may be formulated by further including a pharmaceutically acceptable carrier. The pharmaceutically acceptable carrier refers to a carrier or a diluent that does not cause significant irritation to an organism and does not abrogate the biological activity and properties of the administered component. The pharmaceutically acceptable carriers of the present invention may be physiological saline, sterile water, Ringer's solution, buffered saline, a dextrose solution, a maltodextrin solution, glycerol, ethanol and a combination of one or more thereof. If necessary, other common additives such as an antioxidant, a buffer, a bacteriostatic agent, etc. may be used. Moreover, a diluent, a dispersant, a surfactant, a binder, and a lubricant may be used to prepare injectable formulations such as an aqueous solution, a suspension, an emulsion, etc., pills, capsules, or tablets.

Further, the present invention provides provide a method of preventing or treating a disease by administering the pharmaceutical composition including cells which are surface-modified with the hyaluronic acid derivative or the salt thereof as an active ingredient.

The above descriptions of the hyaluronic acid derivatives or salts thereof may be also applied to the method of preventing or treating a disease.

The disease may be prevented or treated, depending on the therapeutic use of the surface-modified cells, and preferably, the disease may be a liver disease. For example, the liver disease may be one or more selected from the group consisting of liver cancer, metastatic liver cancer, hepatic cirrhosis, hepatitis, and hepatic fibrosis, but are not limited thereto. Specific diseases may be determined depending on the cells surface-modified with the hyaluronic acid derivative or salt thereof.

Still another aspect of the present invention provides a method of modifying the surface of cells using the hyaluronic acid derivative. The method of modifying the surface of cells includes the steps of (1) washing cells with a buffer solution and then treating the cells with trypsin; (2) detaching the cells of step (1) and resuspending the cells in the buffer solution; (3) adding the hyaluronic acid derivative to the resuspended cells of step (2); and (4) mixing the product of step (3), followed by incubation and washing with the buffer solution.

The above descriptions of the hyaluronic acid derivatives or salts thereof may be also applied to the cells surface-modified with the hyaluronic acid derivative or salt thereof, or the method of modifying the surface of cells.

In the method of modifying the surface of cells, the buffer solutions used in step (1), (2) and (4) may be PBS. The buffer solution may be at pH 6 to 8, preferably, at pH 7 to 7.5, or at pH 7.4. For example, the buffer solution used in the present invention may be phosphate or serum-free buffer at pH 7.4.

The hyaluronic acid derivative of step (3) may be added in a state in which the hyaluronic acid derivative is dissolved in an aqueous solution.

The incubation of step (4) may be performed for 0 to 20 minutes, and more preferably, for 5 to 10 minutes.

When WGA is bound to the hyaluronic acid derivative, WGA of the hyaluronic acid derivative may be added at a concentration of 0 to 20 ug/mL, preferably 5 to 10 ug/mL, based on 1X10⁶/mL of cell density. Within the above range of concentration, low cytotoxicity and high liver-targeting efficiency may be achieved.

The cells in the surface modification using the hyaluronic acid derivative according to an embodiment of the present invention may be, are not particularly limited to, cells used as cellular therapeutic agents, and preferably, mammalian cells, mammalian cell-derived cells, or genetically engineered cells thereof, for example, erythrocytes or stem cells, preferably, mesenchymal stem cells.

The hyaluronic acid derivative may be safely applied to the human body, and may be also in a maximal activity state of a protein by reacting with a specific amino acid of the protein, thereby increasing cell surface modification efficiency and achieving liver-targeting delivery. Accordingly, hyaluronic acid derivative may be applied as an effective cell delivery system for the treatment of liver diseases.

The hyaluronic acid derivative capable of modifying the surface of cells has the mucoadhesive property, in addition to its liver-targeting delivery property. Therefore, when delivery of cells to the brain via intranasal injection or delivery of cells to the lung via endobronchial injection is performed, the cells may remain in the target organ for a long time, compared to non-surface modified cells.

### Effect of the Invention

The present disclosure relates to a hyaluronic acid derivative for surface modification of single cells, and a method of preparing the same. When cells surface-coated with the hyaluronic acid derivative may be injected intravenously, target delivery of the cells to the liver may be achieved. The hyaluronic acid derivative capable of modifying the surface of cells and having biocompatibility, biodegradability, and liver-targeting delivery property at the same time may be safely applied to the human body, and also applied to a liver-targeting delivery system of various cells via intravenous injection. Therefore, it is possible to prepare the hyaluronic acid derivative for cell surface modification which may be used in a drug delivery system of cell carrier and tissue engineering, and to provide a liver-targeting cell therapy system via intravenous injection of the cells surface-modified with the hyaluronic acid derivative.

The liver-targeting cell delivery system using the hyaluronic acid derivative of the present invention may be applied as a cellular therapeutic agent for one or more diseases selected from the group consisting of liver cancer, metastatic liver cancer, hepatic cirrhosis, hepatitis and hepatic fibrosis, because the hyaluronic acid derivative may be reacted with cells in an aqueous solution in a simple manner, and a biocompatible and biodegradable polymer, hyaluronic acid derivative is able to deliver cells to the liver due to its liver-targeting delivery property. Further, since this therapeutic agent may deliver stem cells to the liver, it may be utilized in tissue regeneration, such as regeneration of the liver tissue, etc. Accordingly, the cellular therapeutic agent using the hyaluronic acid derivative of the present invention may be efficiently delivered to the liver to show excellent therapeutic effects, upon intravenous injection.

### BRIEF DESCRIPTION OF DRAWINGS

FIG. 1 is a schematic illustration for a liver-targeting delivery system after surface-modification of cells with hyaluronic acid derivatives;

FIG. 2 shows various hyaluronic acid derivatives used in the present invention, in which FIG. 2A shows a lipid-conjugated hyaluronic acid derivative, FIG. 2B shows a 1,6 hexanediamine-maleimide-conjugated hyaluronic acid derivative, FIG. 2C shows a thiol-conjugated hyaluronic acid derivative, and FIG. 2D shows a thiol-conjugated hyaluronic acid derivative further including maleimide-PEG and maleimide-PEG-lipid;

FIG. 3 is a schematic illustration for hyaluronic acid-aldehyde according to an embodiment of the present invention and a reaction process of preparing a hyaluronic acid-protein derivative using the same;

FIG. 4 shows the result of GPC analysis of a WGA-conjugated hyaluronic acid derivative prepared by a method according to Examples of the present invention;

FIG. 5A shows the result of a stem cell (rMSC) toxicity test of the WGA-conjugated hyaluronic acid derivative prepared by a method according to Examples of the present invention;

FIG. 5B shows IC₅₀ values obtained from the results of the toxicity test of FIG. 5A;

FIG. 6 shows the result of a stem cell (hMSC) toxicity test of the WGA-conjugated hyaluronic acid derivative prepared by a method according to Examples of the present invention;

FIG. 7 shows the result of comparing circular dichroism (CD) spectra of WGA and WGA-conjugated hyaluronic acid derivative;

FIG. 8 is a schematic illustration for a method of modifying the surface of cells with the WGA-conjugated hyaluronic acid derivative according to an embodiment of the present invention;

FIG. 9 shows changes in cell viability over time after cell surface modification with the WGA-conjugated hyaluronic acid derivative according to an embodiment of the present invention;

FIG. 10 shows zeta potentials measured after cell surface modification with the WGA-conjugated hyaluronic acid derivative according to an embodiment of the present invention;

FIGS. 11A and 11B show the results of confocal microscopy and flow cytometry after cell surface modification with the WGA-conjugated hyaluronic acid derivative according to an embodiment of the present invention;

FIGS. 12A and 12B show the results of confocal microscopy for cellular uptake over time after surface modification of rMSC and hMSC with the WGA-conjugated hyaluronic acid derivative according to an embodiment of the present invention;

FIGS. 13A and 13B show the results of confocal microscopy and flow cytometry for examining the effect of co-incubation of HA or WGA on the cell binding of the WGA-conjugated hyaluronic acid derivative, upon surface modification with the WGA-conjugated hyaluronic acid derivative;

FIG. 14 shows the result of flow cytometry for examining the cell binding efficiency of the WGA-conjugated hyaluronic acid derivative according to Examples of the present invention or HA;

FIG. 15 shows the result of ELISA assay over time for examining the effect of genetically engineered cells surface-modified with the WGA-conjugated hyaluronic acid derivative according to Examples of the present invention on the release profiles of TRAIL protein;

FIGS. 16A and 16B show a schematic illustration for a transwell coculture assay and MTT assay for examining in-vitro drug effect of the surface-modified, genetically engineering cells on cancer cells;

FIG. 17 shows the result of optical microscopy for examining whether erythrocytes surface-modified with the WGA-conjugated hyaluronic acid derivative according to Examples of the present invention are able to bind to macrophage Raw 264.7;

FIGS. 18A and 18B are ex-vivo images obtained by using an IVIS imaging system, the images showing the phenomena that occurred when the surface-modified cells were intravenously injected to an animal in order to examine the cell targeting effect of the WGA-conjugated hyaluronic acid derivative;

FIG. 19 is a graph showing quantification of ex-vivo images obtained in FIGS. 18A and 18B;

FIG. 20 shows the result of fluorescence microscopy for examining stem cell distribution in the lung and liver dissected in FIGS. 18A and 18B;

FIGS. 21A, 21B, and 21C show a confocal microscopic image of cells co-stained with CFSE and Hilyte 647-labeled WGA-conjugated hyaluronic acid derivatives in order to examine biodistribution of stem cells, and ex-vivo images obtained by using the IVIS imaging system, the images showing the phenomena that occurred when the cells were intravenously injected to an animal;

FIG. 22 is a graph showing quantification of ex-vivo images obtained in FIGS. 21B and 21C;

FIG. 23 shows the result of fluorescence microscopy for examining stem cell distribution in the lung and liver dissected in FIGS. 21B and 21C;

FIG. 24 shows images before merging of the fluorescence microscopic images of the liver of FIG. 23;

FIG. 25 shows confocal microscopic images of colabelling of erythrocytes with Cy5 and FITC-labeled WGA-conjugated hyaluronic acid derivative; and

FIGS. 26A and 26B show in-vivo images over time obtained by using the IVIS imaging system, the images showing the biodistribution that occurred when the colabeled erythrocytes shown in FIG. 25 were intravenously injected to an animal, and a graph showing quantification of the images.

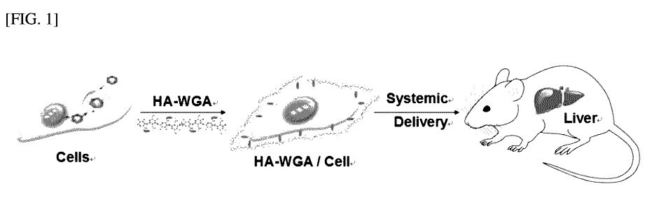

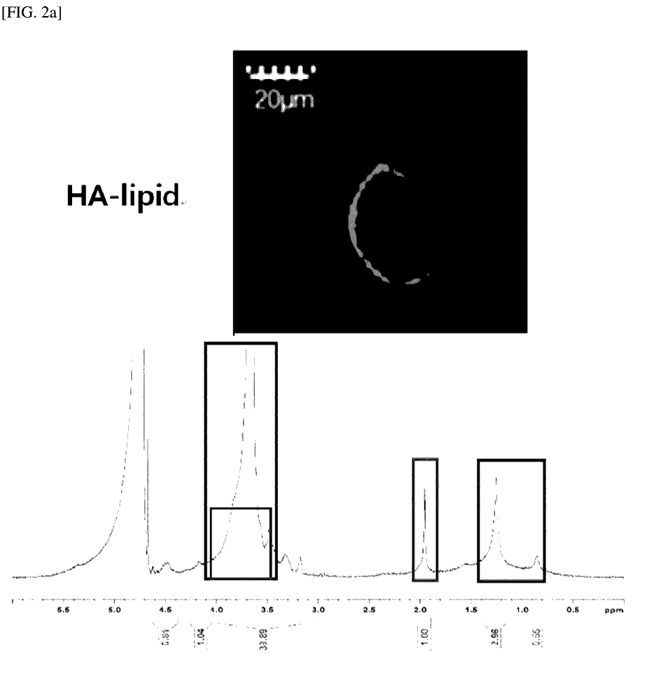

### DETAILED DESCRIPTION OF THE EMBODIMENTS

Hereinafter, various embodiments of the present invention will be described. However, these embodiments are for illustrative purposes only, and the scope of the invention is not intended to be limited thereby.

### Example

### Preparation of WGA—Conjugated Hyaluronic Acid Derivative and Characterization

1-1: Preparation of Hyaluronic Acid-Aldehyde Derivative

Hyaluronic acid (HA) (MW=100 kDa) was dissolved in water at a concentration of 10 mg/ml, and then reacted for 4 hours with the addition of sodium periodate (NaIO4) in an amount of 1 mole times of hyaluronic acid under the dark condition. The reaction product was purified by dialysis against distilled water with a dialysis membrane having a molecular weight cutoff of 7,000 Dalton for 3 days, and then was freeze-dried for 3 days to obtain hyaluronic acid-aldehyde derivatives having the different substitution ratios.

To analyze the substitution ratios of the hyaluronic acid-aldehyde (HA-aldehyde) derivatives, the prepared hyaluronic acid-aldehyde derivatives were dissolved in 100 mM acetate buffer (pH 5.2) at a concentration of 5 mg/ml, and tert-butyl carbazate (TBC) and sodium cyanoborohydride (NaCNBH₃) were added in an amount of 5 mole times with respect to unit of the hyaluronic acid, respectively and reacted for 24 hours to produce hyaluronic acid-aldehyde-TBC, in which TBC was conjugated to the aldehyde group in the hyaluronic acid-aldehyde derivative. The products were subjected to dialysis against distilled water with a dialysis membrane of MWCO 7000 for 3 days, and then were freeze-dried for 3 days to analyze the substitution ratios of aldehyde by 1H-NMR (DPX300, Bruker, Germany)

In the 1H—NMR spectra of the hyaluronic acid-aldehyde-TBC derivatives, 3 methyl peaks of TBC indicating 9 hydrogen atoms appeared at d=1.2˜1.4 ppm, in addition to the peaks of hyaluronic acid. For quantitative analysis, methyl resonance of acetamido moiety of hyaluronic acid at d=1.85˜1.95 ppm was determined as an internal standard.

In the results of 1H—NMR analysis, the peak area of d=1.85˜1.95 ppm and the peak area of d=1.2˜1.4 ppm were compared to calculate the substitution ratio of hyaluronic acid-aldehyde. As a result, the substitution ratio of about 10 mole % was obtained, and thus it was confirmed that hyaluronic acid-aldehyde derivatives having the substitution ratio of about 10 mole % was prepared.

1-2: Preparation of WGA-Conjugated Hyaluronic Acid Derivative

The hyaluronic acid-aldehyde derivative obtained in Example 1-1 was dissolved in 100 mM acetate buffer (pH 5.5) at a concentration of 5 mg/ml, and WGA having an amino acid sequence of SEQ ID NO: 1 was added in an amount of 4 mole times with respect to 1 molecule (chain) of the hyaluronic acid-aldehyde, such that 4 WGAs were bound to 1 molecule (chain) of hyaluronic acid (100 kDa), and reacted at a reaction rate of 95 mole % or more.

According to the substitution ratio of the hyaluronic acid-aldehyde derivative analyzed by the method of Example 1-1, sodium cyanoborohydride (NaCNBH₃) was added in an amount of 5 mole times with respect to 1 mole of aldehyde, and reacted for 24 hours to produce a WGA-conjugated hyaluronic acid derivative. Then, ethyl carbazate was added in an amount of 5 mole times of aldehyde in order to block the unreacted aldehyde group, and further reacted for 24 hours. In this Example, ethyl carbazate was used for blocking, but amino ethanol may be also used. In this case, amino ethanol was added in an amount of 5 mole times of aldehyde, and then pH was increased to 8 using NaOH, and further reacted for 12 hours. This reaction solution was subjected to dialysis against phosphate buffer of pH 7.4, and then stored at −70° C.

1-3. GPC Analysis for WGA Conjugation

Formation of WGA-conjugated hyaluronic acid derivative was confirmed by GPC (Gel Permeation Chromatography) analysis of the WGA-conjugated hyaluronic acid derivative prepared in Example 1-2. GPC analysis of the WGA-conjugated hyaluronic acid derivative was performed using HPLC (High performance liquid chromatography), and the analysis conditions are as follows.

<GPC analysis conditions>

Pump: Waters 1525 binary HPLC pump

Absorbance detector: Waters 2487 dual λ absorbance detector

Sampler: Waters 717 plus auto-sampler

Column: Waters Ultrahydrogel 1000+Waters Ultrahydrogel 500

Mobile phase: PBS (pH 7.4)

Flow rate: 0.4 mL/min

Measurement wavelength: dual detection at 210 nm and 280 nm

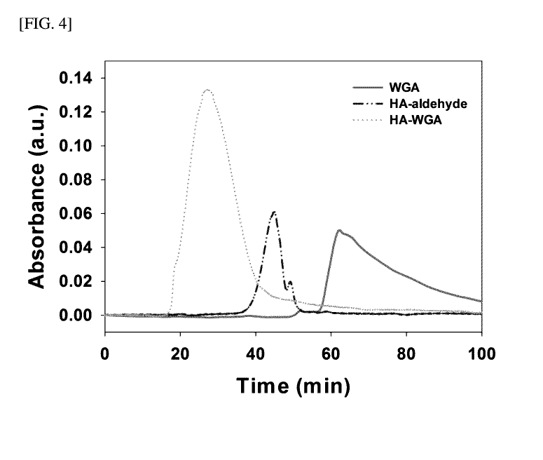

The results of GPC analysis are shown in FIG. 4. FIG. 4 shows the result of GPC analysis of the WGA-conjugated hyaluronic acid derivative prepared by the method according to Examples of the present invention.

As shown in FIG. 4, the peak of WGA appeared at the retention time of 62 min, whereas the peak of WGA-conjugated hyaluronic acid derivative having a high molecular weight appeared at the retention time of 27 min, indicating conjugation of WGA to hyaluronic acid.

1-4: Quantitative Analysis of WGA-Conjugated Hyaluronic Acid Derivative

1 mg/ml of pure WGA solution was diluted at a concentration of 0.5, 1, 5, 10, 20, 30, or 50 μg/ml to prepare a WGA standard solution, and the standard curve was obtained by Bradford assay. That is, the WGA standard solution and a Coomassie protein assay reagent were mixed at a volume ratio of 1:1, and then incubated at room temperature for 10 minutes. Then, absorbance at 595 nm was measured to obtain the standard curve.

The WGA-conjugated hyaluronic acid derivative prepared in Example 1-2 and WGA were diluted 100-fold, and absorbance was measured under the same conditions by Bradford assay, and the absorbance was compared with the standard curve to obtain the content of WGA.

As a result, a ratio of the amount of WGA present in the WGA-conjugated hyaluronic acid derivative to the amount of WGA (feed) added upon synthesis of the WGA-conjugated hyaluronic acid derivative was 95% or more, indicating that the reaction rate of the WGA-conjugated hyaluronic acid derivative was 95 mole % or more.

1-5: CD Analysis of WGA-Conjugated Hyaluronic Acid Derivative

Based on the concentration of WGA (0.25 mg/ml), the WGA solution and the WGA-conjugated hyaluronic acid derivative solution were analyzed by Circular Dichroism (CD). The analysis conditions are as follows.

<CD analysis conditions>

UV spectrophotometer: JASCO J-715

Measuring conditions: 180˜250 nm, N₂ atmosphere

Quartz cuvette: 2 mm path length

Raw data 0 2 mm interval at reaction time of 1 second

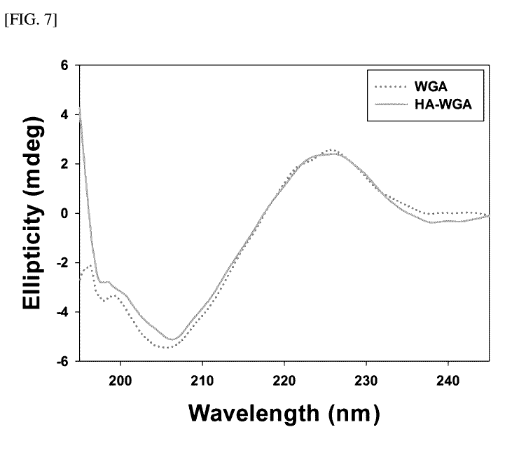

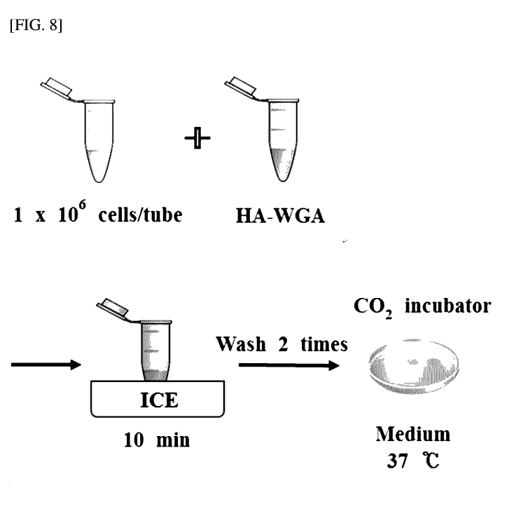

As shown in FIG. 7, it was confirmed that the CD peaks of WGA and WGA-conjugated hyaluronic acid derivative were consistent with each other, and the secondary structure of WGA remained even after the conjugation with hyaluronic acid.

### Example 2

### Analysis of Cell Viability by Cell Surface Modification with WGA-Conjugated Hyaluronic Acid Derivative

2-1. Cell Surface Modification with WGA-Conjugated Hyaluronic Acid Derivative

To confirm cell surface modification by WGA-conjugated hyaluronic acid derivative, the hyaluronic acid derivative of Example 1-2 was labeled with a fluorescent dye FITC, followed by cell surface modification. In detail, a solution, in which the WGA concentration of the WGA-conjugated hyaluronic acid derivative of Example 1-2 was 2 mg/ml, was added in an amount of 5 mole times of FITC, and reacted overnight. To remove FITC which was not bound to the WGA-conjugated hyaluronic acid derivative, a PD 10 column was used. WGA-conjugated hyaluronic acid derivative-FITC thus prepared was used to modify the surface of cells.

To prepare cells surface-modified with the WGA-conjugated hyaluronic acid derivative, the cultured cells were washed with phosphate buffer of pH 7.4, and then detached from a culture dish by treatment with trypsin. The cells were added to phosphate buffer of pH 7.4 at a proper cell density, and resuspended in a microtube. In this regard, as a resuspension solution, serum-free buffer may be also used, in addition to phosphate buffer. To the microtube in which the cells were resuspended, the WGA-conjugated hyaluronic acid derivative solution was added within the range showing no cytotoxicity, and a cell/WGA-conjugated hyaluronic acid derivative mixture was mixed well, and then incubated in ice for 10 minutes to provide a reaction time for the cell and the WGA-conjugated hyaluronic acid derivative. Thereafter, to remove the WGA-conjugated hyaluronic acid derivative that did not bind to the cell surface, the cells were washed with phosphate buffer of pH 7.4. It was confirmed that FITC entered cells, but the WGA-conjugated hyaluronic acid derivative was also bound to cell surface due to WGA binding to the cell surface. In contrast, it was confirmed that hyaluronic acid did not bind to the cell surface.

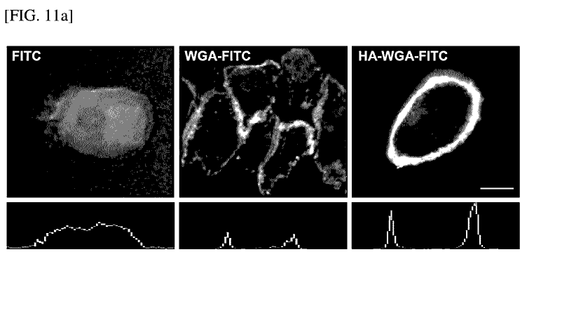

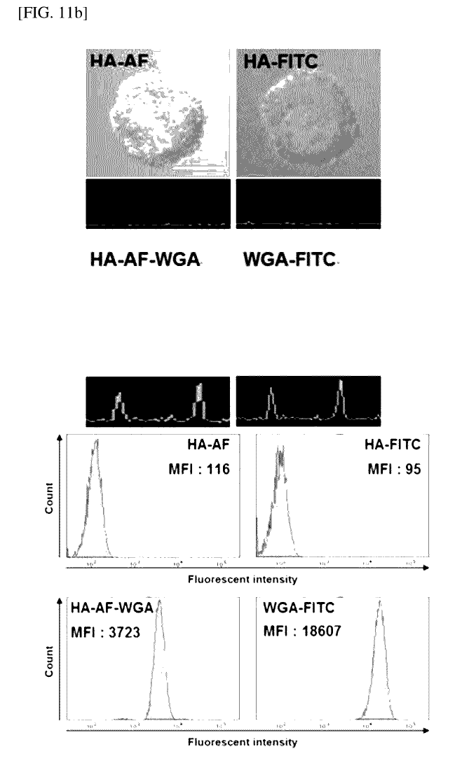

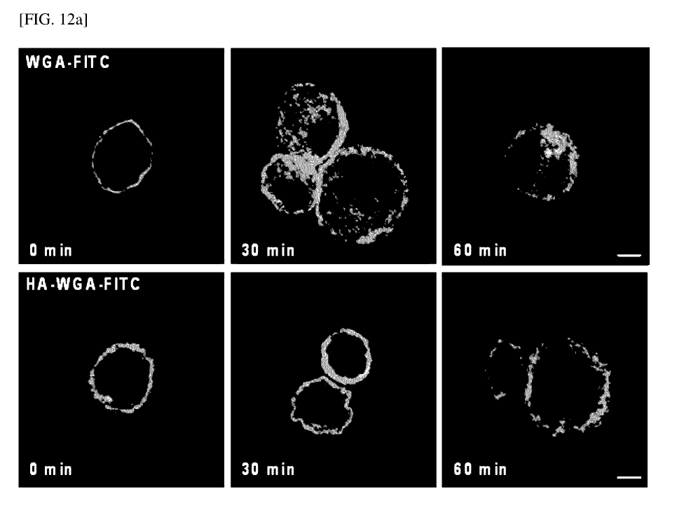

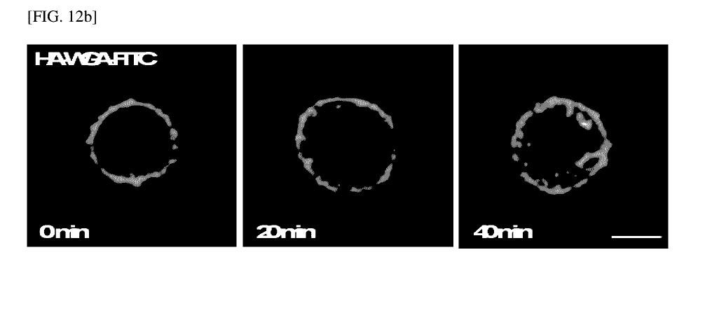

The experimental results are shown in FIGS. 11 and 12. FIG. 11 shows the results of confocal microscopy and flow cytometry after cell surface modification, and FIG. 12 shows the results of confocal microscopy for cellular uptake over time, confirming that the WGA-conjugated hyaluronic acid derivatives were present on the cell surface even after 1 hour.

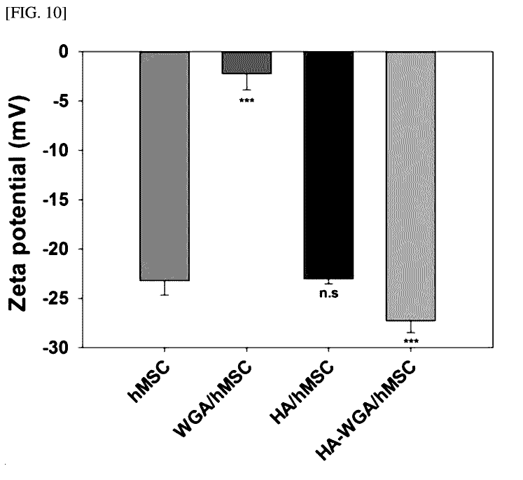

As shown in FIG. 10, when the cell surface was modified with the WGA-conjugated hyaluronic acid derivative, the charge density of hyaluronic acid was very high even though WGA was bound thereto, and therefore, its zeta potential was lower than that of the non-modified control group.

2-2: Analysis of Cytotoxicity of WGA-Conjugated Hyaluronic Acid Derivative

Mesenchymal stem cells were seeded in a 96-well plate at a density of 10⁴ cells/well and cultured for 24 hours. 24 hours later, 1 mg/ml of the WGA solution and the WGA-conjugated hyaluronic acid derivative solution were diluted at various concentrations, based on the concentration of WGA, respectively and each 100 μl of the samples was added to the 96-well plate, followed by incubation for 24 hours.

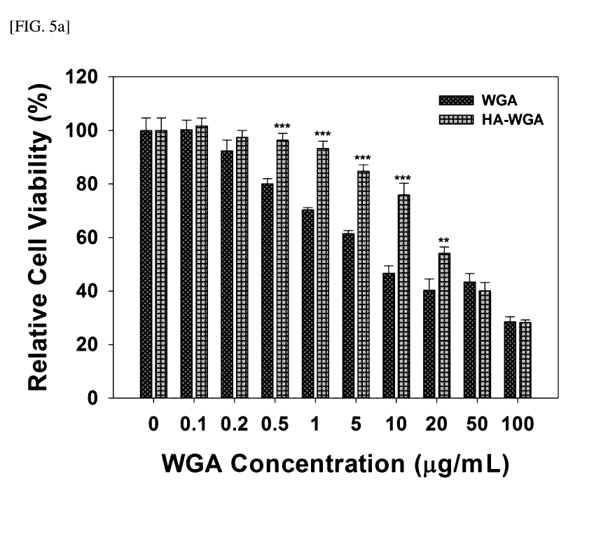

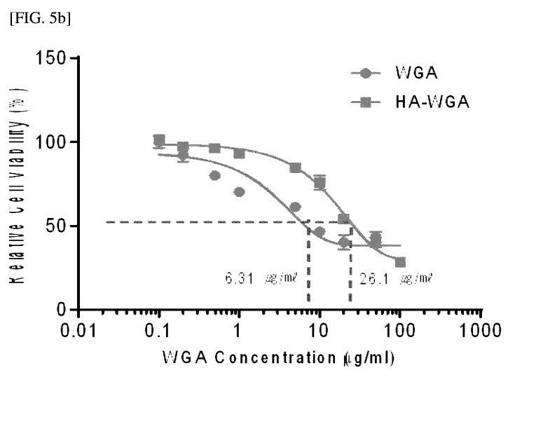

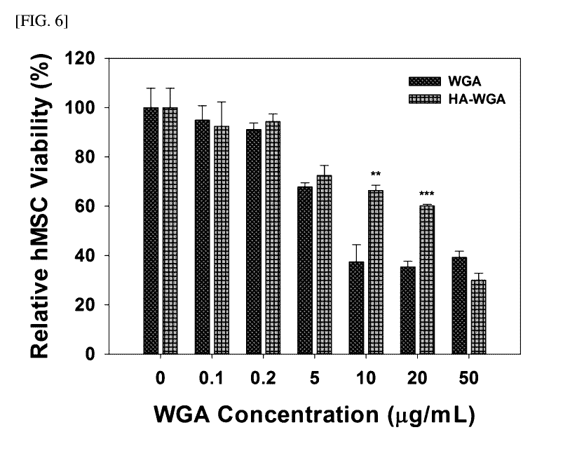

24 hours later, MTT assay was performed to examine the toxicity of the WGA-conjugated hyaluronic acid derivative on cells. The results of the toxicity test are shown in FIGS. 5 and 6. FIG. 5A shows the result of the stem cell (rMSC) toxicity test of the WGA-conjugated hyaluronic acid derivative prepared by a method according to Examples of the present invention, and FIG. 5B shows IC₅₀ values obtained from the results of the toxicity test of FIG. 5A. FIG. 6 shows the result of the stem cell (hMSC) toxicity test of the WGA-conjugated hyaluronic acid derivative prepared by the method according to Examples of the present invention.

As shown in FIGS. 5A and 6, the cytotoxicity of the WGA-conjugated hyaluronic acid derivative were rarely observed at a concentration of 10 μg/ml or less, based on the WGA concentration, and showed lower cytotoxicity than WGA. As shown in FIG. 5B, the IC₅₀ value of the WGA-conjugated hyaluronic acid derivative prepared in Example 1 was found to be 4 times higher than that of WGA.

2-3: Analysis of Cell Viability by Cell Surface Modification

The cell surface was modified with the WGA-conjugated hyaluronic acid derivative of Example 1-2. Surface modification was performed, based on the proper WGA concentration which was determined in Example 2-1.

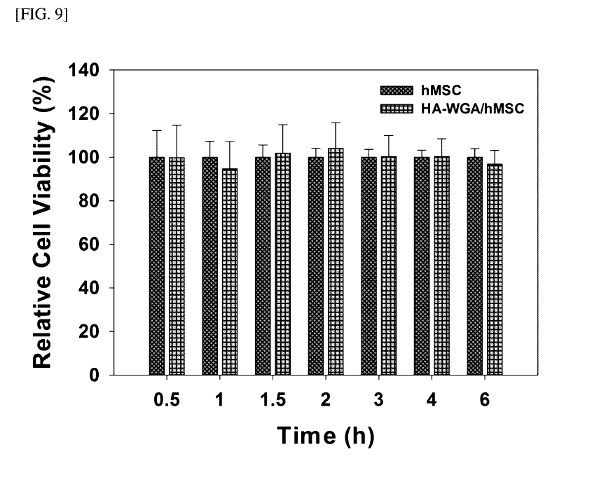

The surface-modified cells were seeded in a 96-well plate at a density of 10⁴ cells/well, and a cell viability test was performed using EZ-Cytox at a predetermined time point (0.5, 1, 1.5, 2, 3, 4, 6 hr). The result is shown in FIG. 9. FIG. 9 shows changes in cell viability over time after cell surface modification with the WGA-conjugated hyaluronic acid derivative according to an embodiment of the present invention.

As shown in FIG. 9, it was confirmed that cell surface modification with the WGA-conjugated hyaluronic acid derivative did not affect cell viability.

2-4: Cell Surface Modification Mechanism of WGA-Conjugated Hyaluronic Acid derivative

5-Aminofluorescein or Hilyte 647 was conjugated to the WGA-conjugated hyaluronic acid derivative in the substantially same manner as in Example 2-1. This conjugate was used to perform a competitive binding test of the WGA-conjugated hyaluronic acid derivative.

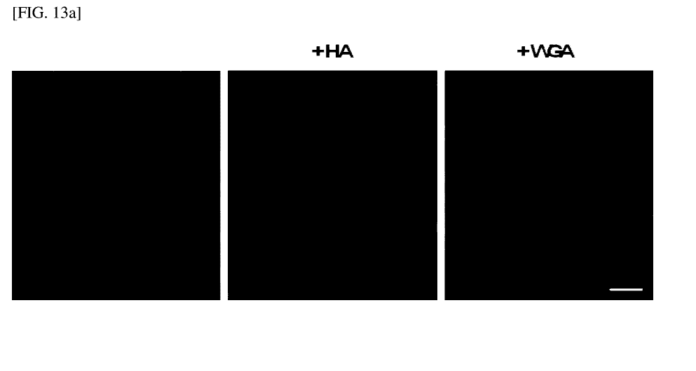

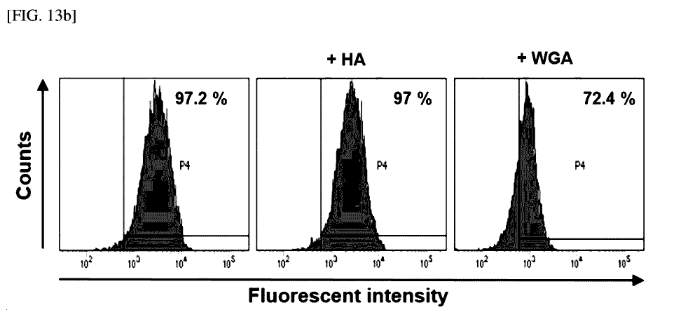

In more detail, when cell surface modification was performed according to Example 2-1, 100 μl of 10 mg/ml 100 kDa HA solution or 50 μl of 2 mg/ml WGA solution were added together with the WGA-conjugated hyaluronic acid derivative to perform surface modification. Confocal microscopy and flow cytometry were used to examine binding of the WGA-conjugated hyaluronic acid derivatives and cells. The experimental results are shown in FIGS. 13A and 13B. FIGS. 13A and 13B show the results of confocal microscopy and flow cytometry for examining the effect of co-incubation of free HA or free WGA on the cell binding of the WGA-conjugated hyaluronic acid derivative, upon surface modification with the WGA-conjugated hyaluronic acid derivative.

As shown in FIGS. 13A and 13B, the highest fluorescence intensity was observed in the cells surface-modified without the competitive binding material, and the lowest fluorescence intensity was observed in the cells surface-modified with addition of free WGA. Further, the result of flow cytometry showed that the binding efficiency of the WGA-conjugated hyaluronic acid derivative to cells surface-modified together with free HA was 97%, which was higher than that of the cells surface-modified together with free WGA (72.4%). That is, it can be seen that cell surface modification occurs by binding of WGA present in the WGA-conjugated hyaluronic acid derivative mainly to sialic acid.

2-5: Cell Surface Binding Efficiency of WGA-Conjugated Hyaluronic Acid Derivative

To examine cell surface binding efficiency of hyaluronic acid and WGA-conjugated hyaluronic acid derivative, flow cytometry was used. In more detail, Hilyte 647 amine was labeled to hyaluronic acid-aldehyde prepared in Example 1. WGA was added to the half thereof to prepare a Hilyte 647-labeled WGA-conjugated hyaluronic acid derivative according to Example 1. Hilyte 647-labeled hyaluronic acid and Hilyte 647-labeled WGA-conjugated hyaluronic acid derivative were used, based on the concentration of HA (50, 25, 12.5, 6.25 μg/ml), to perform cell surface modification according to Example 2, and cell surface binding efficiency was examined by flow cytometry. FIG. 14 shows the result of flow cytometry for examining the cell binding efficiency of the WGA-conjugated hyaluronic acid derivative according to Examples of the present invention or HA.

As shown in FIG. 14, it was confirmed that the WGA-conjugated hyaluronic acid derivative showed more efficient cell surface modification than hyaluronic acid, based on the same concentration of HA.

TABLE 1

HA concentration (μg/ml)

| Section | 50 | 25 | 12.5 | 6.25 |
|---|---|---|---|---|
| HA | 5.2% | 2.1% | 0.5% | 0.5% |
| HA-WGA | 95.1% | 93.6% | 91.2% | 89.6% |

### Example 3

### Effect of WGA-Conjugated Hyaluronic Acid Derivative on Genetically Engineered Cells

The surface of cells, which were genetically engineered to release TRAIL protein, was modified with the WGA-conjugated hyaluronic acid derivative, and the effect of modification on TRAIL release profiles was examined by an ELISA assay.

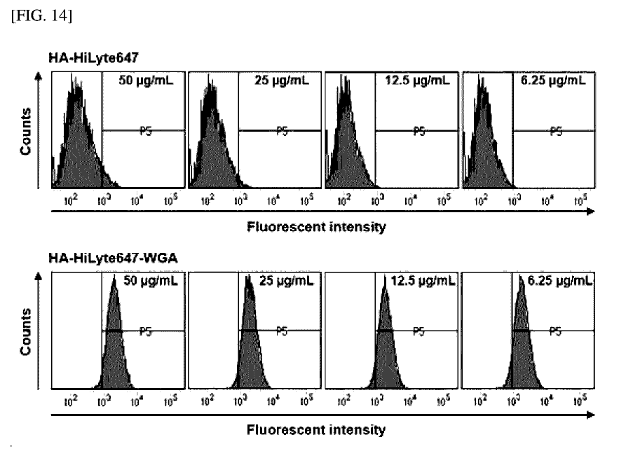

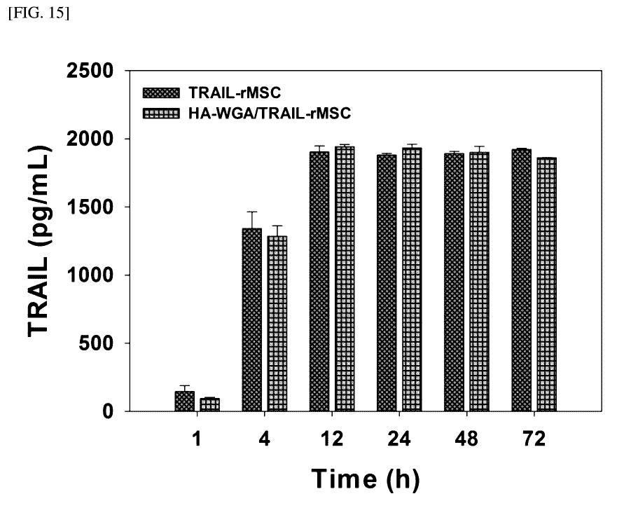

Genetically engineered cells (rat bone marrow-derived mesenchymal stem cells genetically modified to release TRAIL protein, provided by Prof. Young-Chul Sung, Division of Molecular and Life Sciences, Pohang University of Science and Technology) and the WGA-conjugated hyaluronic acid derivative prepared in Example 1-2 were added to a transwell cell insert and incubated to modify the surface of the genetically engineered cells with the hyaluronic acid derivative. At a predetermined time (culture time: 1, 4, 12, 24, 48, 72 hrs), cell culture was extracted. Analysis of the cell culture was performed using a TRAIL ELISA kit, and the result is shown in FIG. 15. FIG. 15 shows the result of ELISA assay over time for examining the effect of genetically engineered cells surface-modified with the WGA-conjugated hyaluronic acid derivative according to Examples of the present invention on the release profiles of TRAIL protein.

As shown in FIG. 15, the genetically engineered cells surface-modified with the WGA-conjugated hyaluronic acid derivative showed the TRAIL protein release pattern similar to that of non-surface modified, genetically engineered cells, and thus it was confirmed that cell surface modification does not affect TRAIL protein release.

### Example 4

### In-Vitro Therapeutic Effect of Genetically Engineered Cells Surface-Modified with WGA-Conjugated Hyaluronic Acid Derivative

In order to examine in-vitro therapeutic effect of genetically engineered cells surface-modified with the WGA-conjugated hyaluronic acid derivative on cancer cells, a transwell co-culture assay was performed.

A hepatoma cell HepG2 was seeded in a 24-well plate at a density of 1×10⁵ cells/well. 24 hours later, the genetically engineered cells were seeded on the upper side of transwell at a density of 10⁵ cells/well, followed by incubation for 72 hours (FIG. 15A).

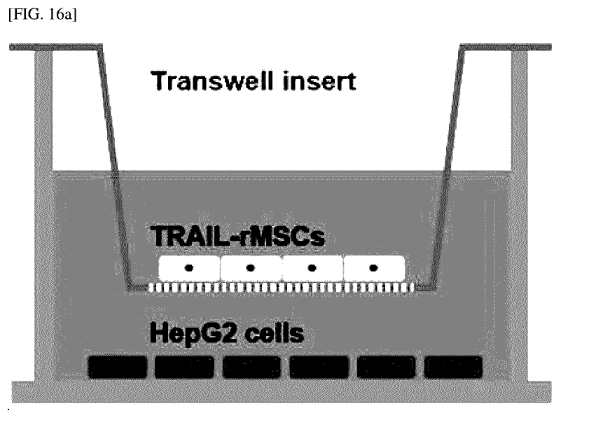

As the genetically engineered cells (rat bone marrow-derived mesenchymal stem cells genetically modified to release TRAIL protein, provided by Prof. Young-Chul Sung, Division of Molecular and Life Sciences, Pohang University of Science and Technology), surface-modified cells by the method of Example 2 (HA/TRAIL-rMSC) were used. As control groups, non-surface-modified cells (TRAIL-rMSC) and rMSC were used. Because rMSC itself may exhibit the therapeutic effect, rMSC was used as the control group of TRAIL-rMSC. Thereafter, HepG2 was subjected to an MTT assay to perform a cytotoxicity test. A schematic illustration for a transwell coculture assay and the result of MTT assay for in-vitro drug activity of the surface-modified genetically engineering cells on cancer cells are shown in FIG. 16.

As shown in FIG. 16, it can be seen that the genetically engineering cells surface-modified with the WGA-conjugated hyaluronic acid derivative exhibit sufficient therapeutic effects on cancer cells.

### Example 5

### In-Vitro binding of Cells Surface-Modified with WGA-Conjugated Hyaluronic Acid Derivative

In order to examine in-vitro binding of erythrocytes surface-modified with the WGA-conjugated hyaluronic acid derivative to macrophage Raw 264.7, an optical microscope was used.

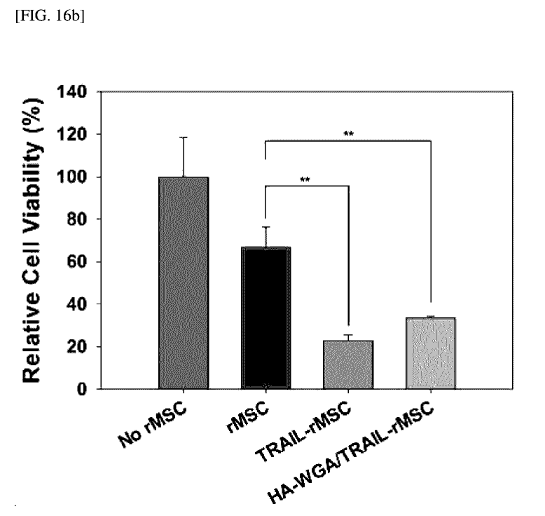

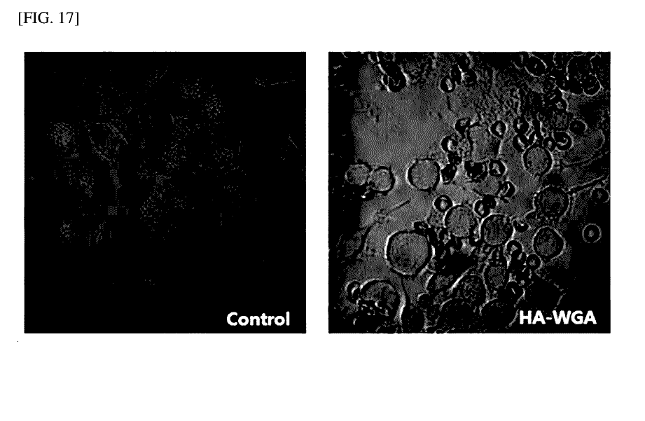

Erythrocytes extracted from a human were surface-modified by the method of Example 2-1 to prepare erythrocytes surface-modified with the WGA-conjugated hyaluronic acid derivative. The erythrocytes surface-modified with the WGA-conjugated hyaluronic acid derivative were added to a cell culture dish on which macrophage Raw 264.7 was seeded. 10 minutes later, to remove erythrocytes which were not bound to the macrophage, the dish was washed with phosphate buffer. The optical microscope was used to examine whether the erythrocytes surface-modified with the WGA-conjugated hyaluronic acid derivative bind to macrophage Raw 264.7, and the result is shown in FIG. 17.

As shown in FIG. 17, it was confirmed that non-surface modified erythrocytes did not bind to macrophage whereas the erythrocytes surface-modified with the WGA-conjugated hyaluronic acid derivative bound to macrophage due to hyaluronic acid.

### Example 6

### Liver-Targeting (Biodistribution) of Stem Cells Surface-Modified with WGA-Conjugated Hyaluronic Acid Derivative

In-vivo body distribution of stem cells surface-modified with the WGA-conjugated hyaluronic acid derivative was examined by using an IVIS imaging system and a fluorescence microscope.

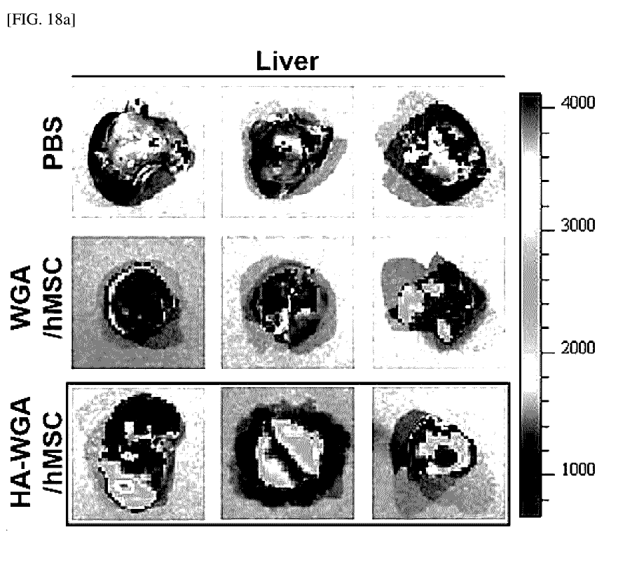

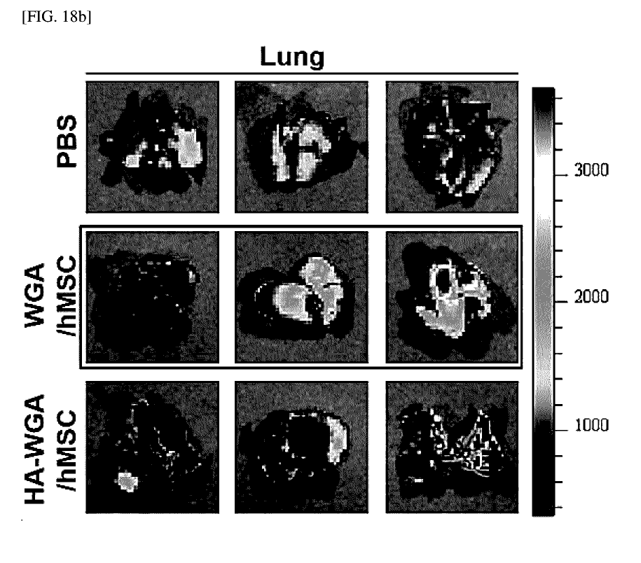

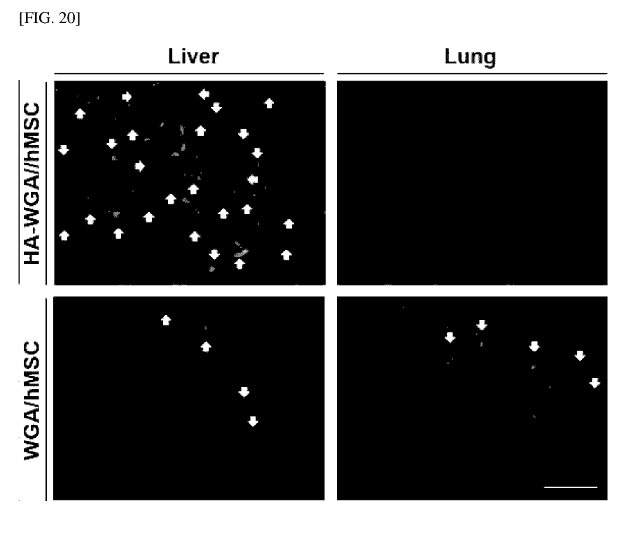

First, it was examined that the liver-targeting delivery of stem cells surface-modified with the WGA-conjugated hyaluronic acid derivative is attributed to hyaluronic acid. Stem cells surface-modified with WGA-FITC and stem cells surface-modified with HA-WGA-FITC were intravenously injected to rats at a cell density of 10⁶ cells per rat, respectively. 4 hours later, the rats were sacrificed and ex-vivo imaging was performed using the IVIS imaging system (FIG. 18). Furthermore, the removed lung and liver were dissected and a fluorescence microscope was used to examine stem cells distributed in the lung and liver tissues. The results are shown in FIG. 20.

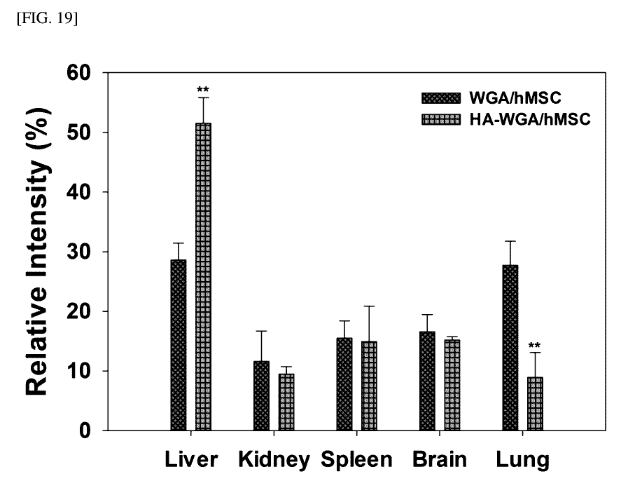

FIGS. 18A and 18B are ex-vivo images obtained by using the IVIS imaging system, the images showing the phenomena that occurred upon intravenous injection of cells to an animal. FIG. 19 is a graph showing quantification of ex-vivo images obtained in FIGS. 18A and 18B. FIG. 20 shows the result of fluorescence microscopy for examining stem cell distribution in the lung and liver dissected in FIGS. 18A and 18B;

As a result, the stem cells surface-modified with WGA-FITC showed similar florescence intensity in the liver and lung, whereas the stem cells surface-modified with the WGA-conjugated hyaluronic acid derivative showed higher florescence intensity in the liver than in the lung (FIGS. 18 and 19). The result of examining the dissected lung and liver tissues under a fluorescence microscope also showed the same patterns as above (FIG. 20). Therefore, it was confirmed that surface modification of the stem cells with the WGA-conjugated hyaluronic acid derivative provides the liver-targeting capability due to hyaluronic acid.

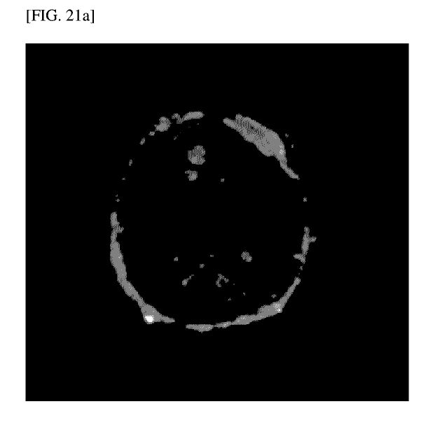

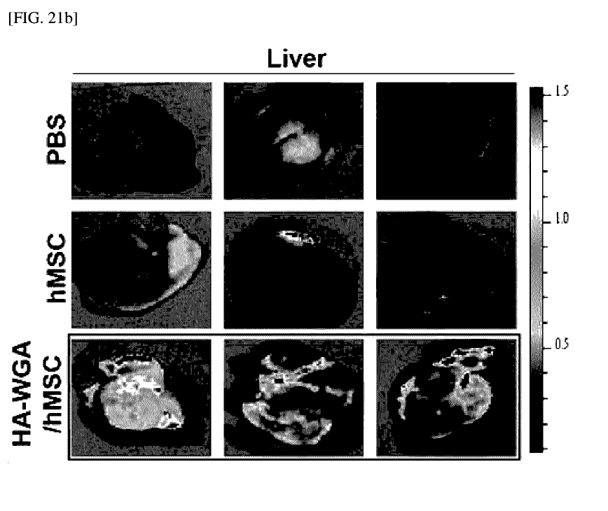

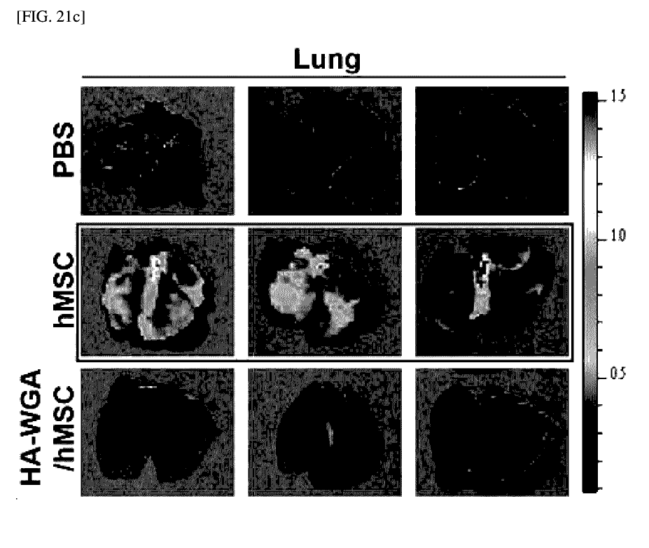

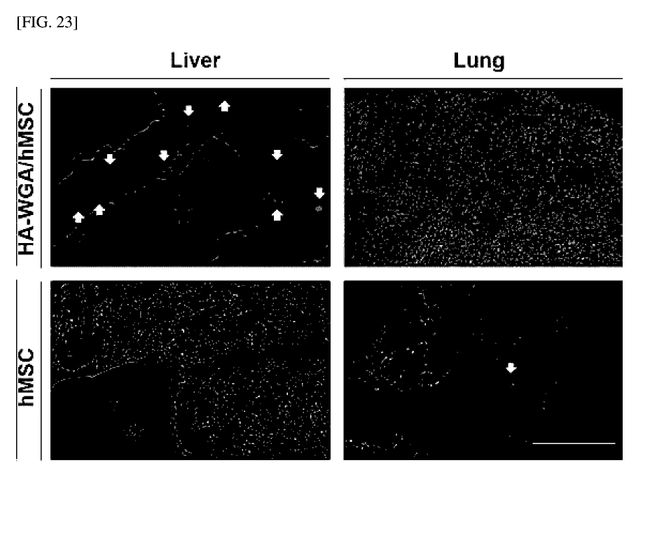

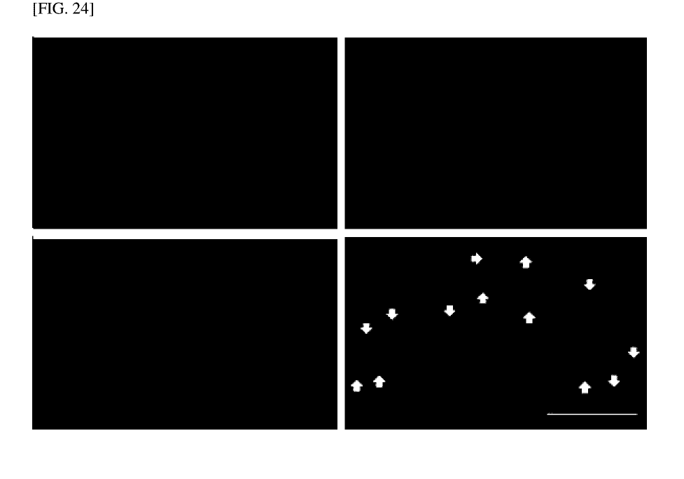

Second, in-vivo body distribution of surface-modified stem cells and WGA-conjugated hyaluronic acid derivative was examined. According to the preparation method of the FITC-labeled WGA-conjugated hyaluronic acid derivative of Example 2-1, Hilyte 647 NHS ester was used to prepare Hilyte 647-labeled WGA-conjugated hyaluronic acid derivative. Stem cells (purchased from Neuromics Co., Ltd.) were first stained with CFSE (sigma). Half of the stem cells thus stained was surface-modified with Hilyte 647-labeled WGA-conjugated hyaluronic acid derivative (FIG. 21A). The surface-modified stem cells were intravenously injected to rats at a cell density of 10⁶ cells per rat. 4 hours later, the rats were sacrificed and ex-vivo imaging was performed using the IVIS imaging system (FIGS. 21B and 21C). Furthermore, the removed lung and liver were dissected and a fluorescence microscope was used to examine stem cells distributed in the lung and liver tissues. The results are shown in FIGS. 23 and 24. The cells modified with only hyaluronic acid without WGA were excluded in-vivo, because the cells were considered to have low cell surface binding capability on in-vitro experiments (e.g., flow cytometry).

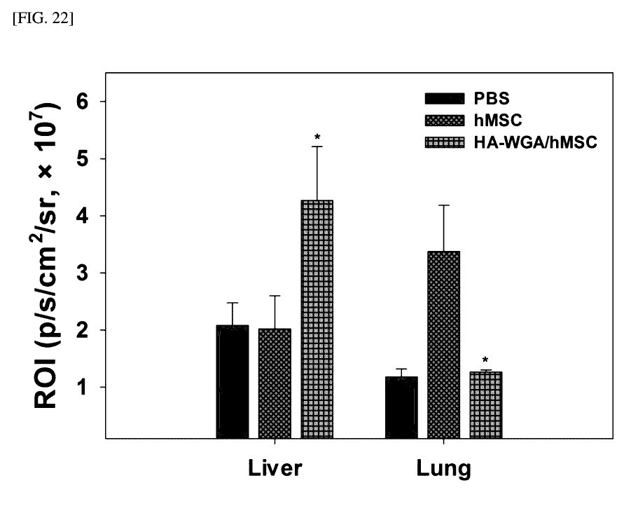

FIG. 21A shows a confocal microscopic image of cells co-stained with CFSE and Hilyte 647-labeled WGA-conjugated hyaluronic acid derivatives in order to examine biodistribution of stem cells, and FIGS. 21B, and 21C and ex-vivo images obtained by using the IVIS imaging system, the images showing the phenomena that occurred upon intravenous injection of cells to an animal. FIG. 22 is a graph showing quantification of ex-vivo images obtained in FIGS. 21B and 21C. FIG. 23 shows the result of fluorescence microscopy for examining stem cell distribution in the lung and liver dissected in FIGS. 21B and 21C. FIG. 24 shows images before merging of the fluorescence microscopic images of the liver of FIG. 23.

As a result, non-surface modified stem cells were mainly found in the lung, and the stem cells surface-modified with the WGA-conjugated hyaluronic acid derivative were mainly found in the liver (FIGS. 21 and 22). The result of examining dissected liver tissues under a fluorescence microscope showed the colocalization of CFSE signal from stem cells and Hilyte 647 signal from Hilyte 647-labeled WGA-conjugated hyaluronic acid derivative (FIGS. 23 and 24). Accordingly, it was confirmed that the liver-targeting capability is generated by surface modification of stem cells with the WGA-conjugated hyaluronic acid derivative.

### Example 7

### Liver-Targeting of Erythrocytes Surface-Modified with WGA-Conjugated Hyaluronic Acid Derivative

Erythrocytes were used to examine biodistribution in BALB/c mice, like in the stem cells.

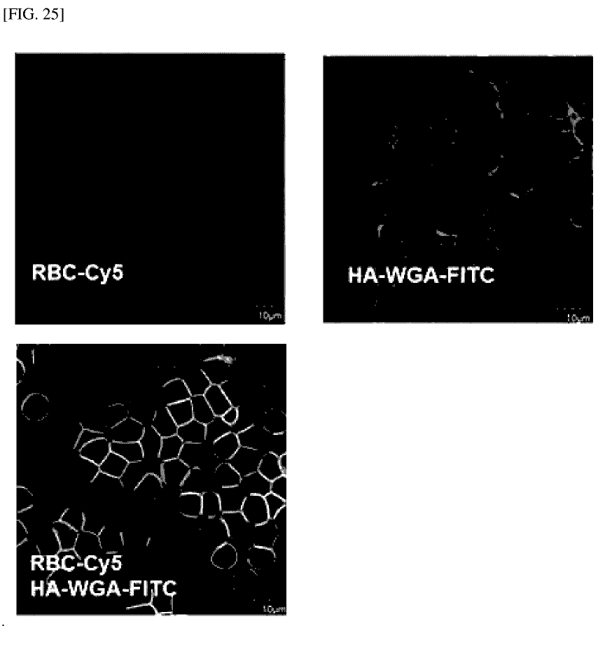

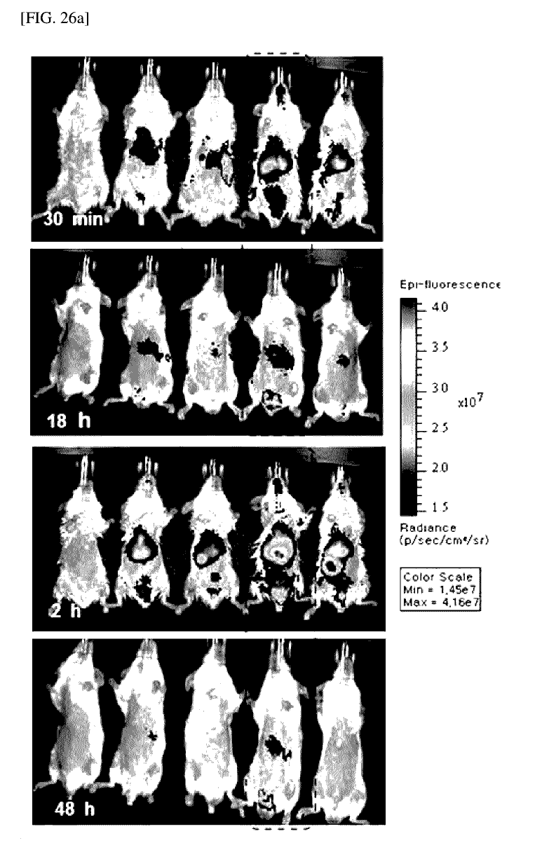

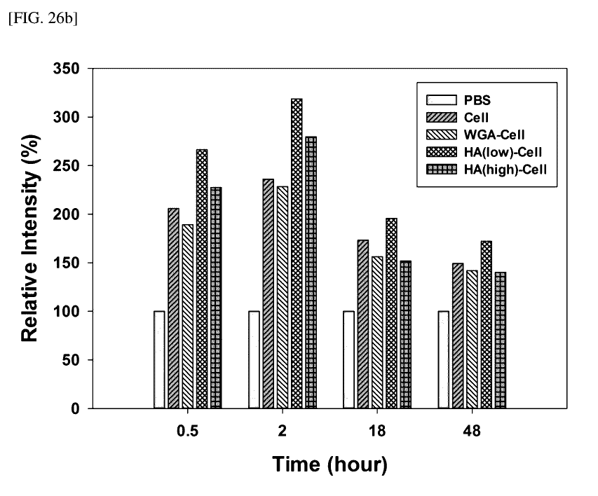

To this end, the surface of erythrocyte was stained with Cy5(lumiprobe), and additionally surface-modified with FITC(sigma)-labeled WGA-conjugated hyaluronic acid derivative. The resulting cells were examined under a confocal microscope (FIG. 25), and the erythrocytes thus surface-modified were intravenously injected to mice. In-vivo imaging was performed using the IVIS imaging system at a predetermined time point (0.5, 2, 18, 48 hr), and the result is shown in FIG. 26. The hyaluronic acid aldehyde derivative with a high aldehyde substitution ratio was indicated by HA(high) and the hyaluronic acid aldehyde derivative with a low aldehyde substitution ratio was indicated by HA(low).

As a result, the confocal microscopy showed successful colabelling of Cy5 and FITC-labeled WGA-conjugated hyaluronic acid derivatives on the surface of erythrocytes (FIG. 25). Further, as shown in FIG. 26, erythrocytes surface-modified with the WGA-conjugated hyaluronic acid derivative having the most excellent liver-targeting capability remain abundant in the liver for a long time.

### Example 8

### Preparation of Lipid-Conjugated Hyaluronic Acid Derivative (HA-lipid)

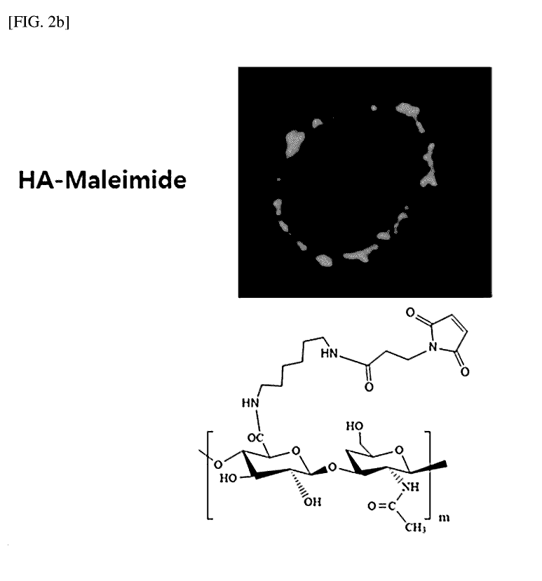

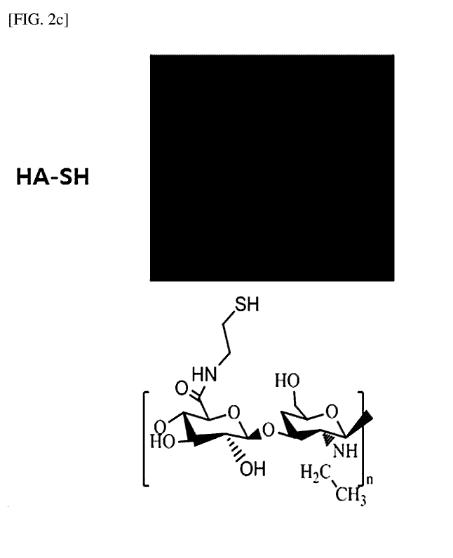

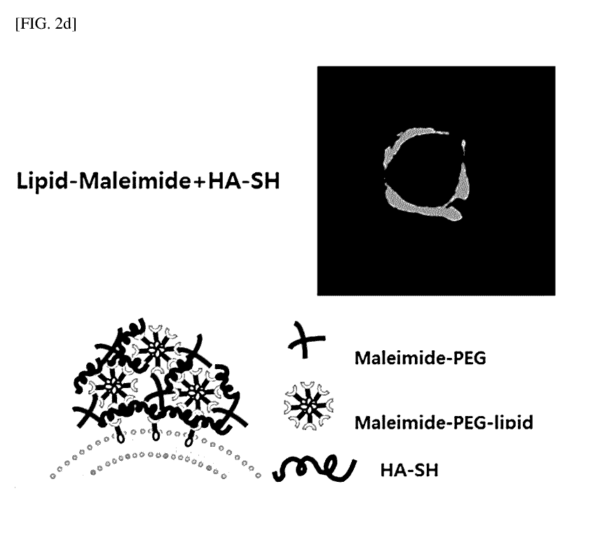

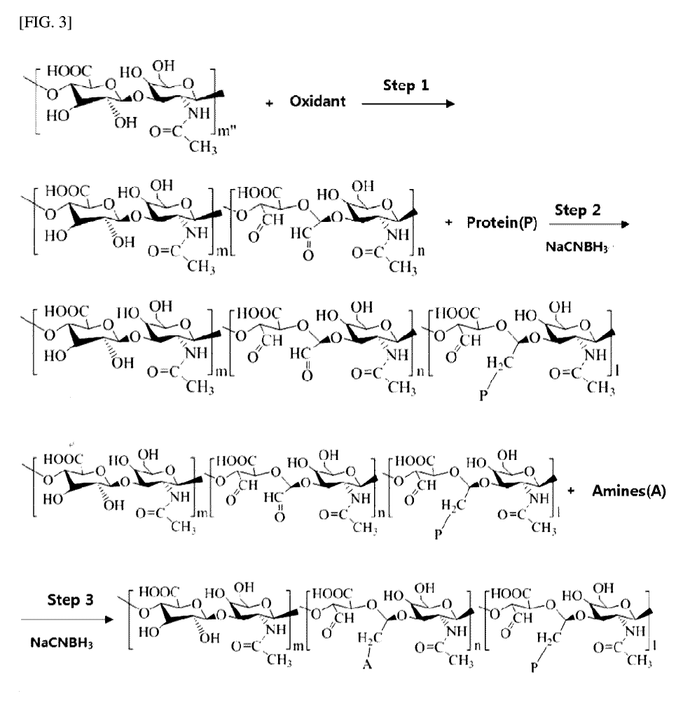

HA having a molecular weight of about 100 kDa was used to prepare a HA aqueous solution of which concentration becomes 5 mg/ml upon reaction. NH₂-PEG-lipid was added thereto in an amount of 5 times the equivalent weight of HA, and completely dissolved under stirring. EDC was added thereto in an amount of 4 times the equivalent weight of HA, and completely dissolved under stirring. Sulfo—NHS was added thereto in an amount equal to the equivalent weight of EDC, and dissolved under stirring. 1 N HCl was added to the resulting aqueous solution, and allowed to react for 6 hours while pH was maintained at 6. 6 hours later, to terminate the reaction, 1 N NaOH was added to increase pH to 7.4. The resulting product was put in a dialysis tube (MW 7000 Da), and subjected to dialysis against 100 mM NaCl aqueous solution for 60 hours. Thereafter, the product was repeatedly subjected to dialysis against 25% ethanol and distilled water, respectively. The aqueous solution from which impurities were removed by dialysis was freeze-dried for 3 days to obtain a pure HA-lipid derivative. The result of conjugating COOH present in HA with NH₂-PEG-lipid by EDC/sulfo—NHS chemistry is shown in FIG. 2A.

### Example 9

### Preparation of Maleimide-Conjugated Hyaluronic Acid Derivative (HA-maleimide)

HA having a molecular weight of about 100 kDa was used to prepare a HA aqueous solution of which concentration becomes 5 mg/ml upon reaction. Hexamethylene diamine, one of diamines, was added thereto in an amount of 10 times the equivalent weight of HA, and completely dissolved under stirring. EDC was added thereto in an amount of 4 times the equivalent weight of HA, and completely dissolved under stirring. Sulfo—NHS was added thereto in an amount equal to the equivalent weight of EDC, and dissolved under stirring. 1 N HCl was added to the resulting aqueous solution, and allowed to react for 2 hours while pH was maintained at 6. 2 hours later, to terminate the reaction, 1 N NaOH was added to increase pH to 7.4. The resulting product was put in a dialysis tube (MW 7000 Da), and subjected to dialysis against 100 mM NaCl aqueous solution for 60 hours. Thereafter, the product was repeatedly subjected to dialysis against 25% ethanol and distilled water, respectively. The aqueous solution from which impurities were removed by dialysis was freeze-dried for 3 days to obtain a pure HA-HMDA derivative as a building block of HA-maleimide.

3-Maleimidopropionic acid was added to HA-HMDA in an amount of 10 times the equivalent weight of HA, and completely dissolved under stirring. EDC was added thereto in an amount of 4 times the equivalent weight of HA, and completely dissolved under stirring. Sulfo—NHS was added thereto in an amount equal to the equivalent weight of EDC, and dissolved under stirring. 1 N HCl was added to the resulting aqueous solution, and allowed to react for 2 hours while pH was maintained at 6. 2 hours later, to terminate the reaction, 1 N NaOH was added to increase pH to 7.4. The resulting product was put in a dialysis tube (MW 7000 Da), and subjected to dialysis against 100 mM NaCl aqueous solution for 60 hours. Thereafter, the product was repeatedly subjected to dialysis against 25% ethanol and distilled water, respectively. The aqueous solution from which impurities were removed by dialysis was freeze-dried for 3 days to obtain a maleimide-introduced hyaluronic acid derivative.

An amine group-induced HA conjugate was prepared by conjugating COOH present in HA with the amine group present in hexamethylendiamine by EDC/sulfo—NHS chemistry. To this conjugate, 3-maleimidopropionic acid was conjugated by EDC/sulfo—NHS chemistry to induce a maleimide group. The result is shown in FIG. 2B.

### Example 10

### Preparation of Thiol-Conjugated Hyaluronic Acid Derivative (HA-SH)

First, cystamine was introduced to HA by EDC/sulfo—NHS chemistry, and then treated with DTT or TCEP to break S-S bonds for 1˜2 hours. 200 mg of hyaluronic acid (HA) (MW=100 kDa) was dissolved in 20 ml of distilled water, and then 1-ethyl-3-(3-dimethylaminopropyl)carbodiimide (EDC) and Sulfo—NHS were added in a molar ratio of 4 times with respect to the carboxyl group of HA, and activated for 30 minutes. Thereafter, cystamine dihydrochloride was added in a molar ratio of 20 times with respect to the carboxyl group of HA, and pH of the solution was adjusted to 4.8, and allowed to react for overnight. After termination of the reaction, from the solution, impurities were removed by ethanol precipitation and remaining precipitates were dissolved in 20 ml of distilled water. Tris(2-carboxyethyl)phosphine (TCEP) was added in a molar ratio of 4 times with respect to the carboxyl group of HA, and the pH of the solution was adjusted to 6, and then allowed to react for 4 hours. After termination of the reaction, from the solution, impurities were removed by ethanol precipitation and remaining precipitates were dissolved in 20 ml of distilled water, and freeze-dried for 3 days to obtain HA-SH. The result of confocal microscopy of the prepared HA-SH is shown in FIG. 2C.

1. A cell surface-modification composition, comprising a hyaluronic acid derivative comprising units represented by the following Chemical Formulae 1 to 3 or a salt thereof; or
a hyaluronic acid derivative comprising units represented by the following Chemical Formulae 1 and 4 or a salt thereof: 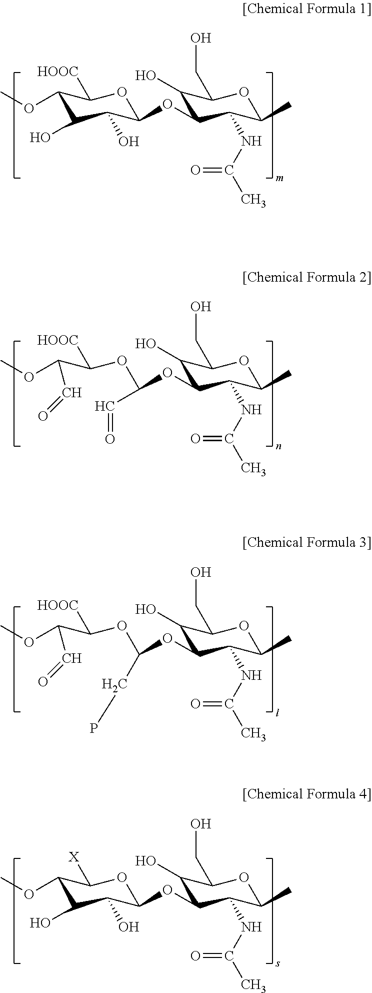
wherein P is a water-soluble protein;
X is —COR¹, —CONR²H—R¹ or —CONH—R²—NH—R¹;
R¹ is a vinyl group, a thiol group, poly-L-lysine, a lipid, an acrylate group, a maleimide group, or N-hydroxysuccinimide (NHS);
R² is an alkyl group having 1 to 6 carbon atoms; and
the sum of m, n, and 1 is an integer of 50 to 10,000, the sum of n and 1 is an integer of 5 to 5,000, 1 is an integer of 1 to 100, and s is an integer of 1 to 2000.

2. The composition of claim 1, wherein the water-soluble protein is a wheat germ agglutinin protein.

3. The composition of claim 1, wherein X is —COR¹, R¹ is a lipid or a thiol group, and the sum of m and s is an integer of 50 to 10,000.

4. The composition of claim 1, wherein X is —CONH—R²—NH—R¹, R¹ is maleimide, R² is a hexyl group, and the sum of m and s is an integer of 50 to 10,000.

5. The composition of claim 1, further comprising maleimide, maleimide-PEG or a composite chemically conjugated maleimide-PEG and lipid.

6. The composition of claim 1, wherein the hyaluronic acid, or the salt of the hyaluronic acid has a molecular weight of 10,000 to 3,000,000 Dalton (Da).

7. The composition of claim 1, wherein the composition delivers cells to the liver by modifying the surfaces of cells.

8. The composition of claim 1, wherein the cells are one or more selected from the group consisting of erythrocytes, stem cells, and genetically engineered cells thereof.

9. The composition of claim 1, comprising a hyaluronic acid derivative represented by the following Chemical Formula 5 or a salt thereof: 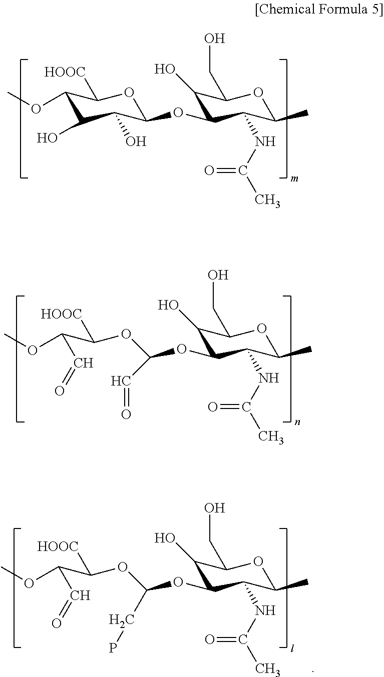
wherein P is a water-soluble protein; and
the sum of m, n and 1 is an integer of 50 to 10,000, the sum of n and 1 is an integer of 5 to 5,000, and 1 is an integer of 1 to 100.

10. The composition of claim 1, comprising a hyaluronic acid derivative represented by the following Chemical Formula 6 or a salt thereof: 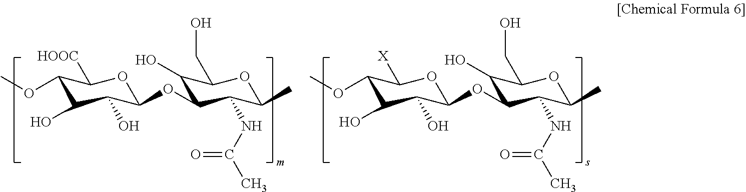
wherein X is —COR¹, —CONR²H—R1 or —CONH—R²—NH—R¹;
R¹ is a vinyl group, a thiol group, poly-L-lysine, a lipid, an acrylate group, a maleimide group, or N-hydroxysuccinimide (NHS);
R² is an alkyl group having 1 to 6 carbon atoms; and
the sum of m and s is an integer of 50 to 10,000, and s is an integer of 1 to 2000.

11. A pharmaceutical composition for the prevention or treatment of liver diseases, comprising surface-modified cells as an active ingredient, wherein the cell surface is modified with a hyaluronic acid derivative comprising units represented by the following Chemical Formulae 1 to 3 or a salt thereof; or
a hyaluronic acid derivative comprising units represented by the following Chemical Formulae 1 and 4 or a salt thereof: 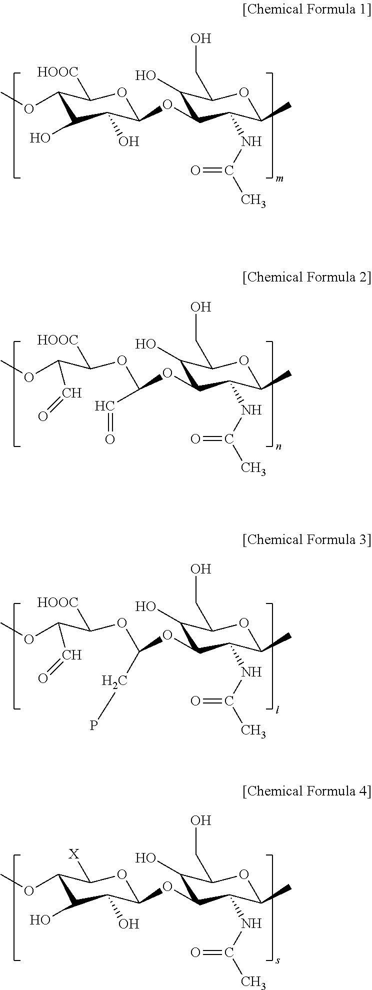
wherein P is a water-soluble protein;
X is —COR¹, —CONR²H—R¹ or —CONH—R²—NH—R¹;
R¹ is a vinyl group, a thiol group, poly-L-lysine, a lipid, an acrylate group, a maleimide group, or N-hydroxysuccinimide (NHS);
R² is an alkyl group having 1 to 6 carbon atoms; and
the sum of m, n, and 1 is an integer of 50 to 10,000, the sum of n and 1 is an integer of 5 to 5,000,1 is an integer of 1 to 100, and s is an integer of 1 to 2000.

12. A method of preventing or treating liver diseases by administering a pharmaceutical composition comprising surface-modified cells as an active ingredient, wherein the cell surface is modified with a hyaluronic acid derivative comprising units represented by the following Chemical Formulae 1 to 3 or a salt thereof; or a hyaluronic acid derivative comprising units represented by the following Chemical Formulae 1 and 4 or a salt thereof: 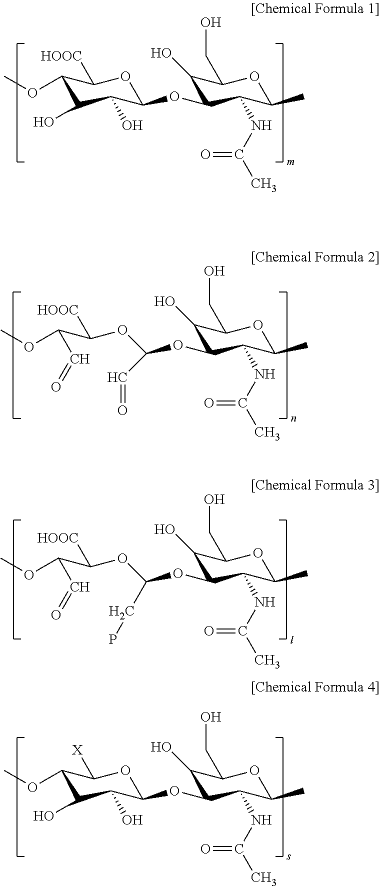
wherein P is a water-soluble protein;
X is —COR¹, —CONR²H—R¹ or —CONH—R²—NH—R¹;
R¹ is a vinyl group, a thiol group, poly-L-lysine, a lipid, an acrylate group, a maleimide group, or N-hydroxysuccinimide (NHS);
R² is an alkyl group having 1 to 6 carbon atoms; and
the sum of m, n, and 1 is an integer of 50 to 10,000, the sum of n and 1 is an integer of 5 to 5,000, 1 is an integer of 1 to 100, and s is an integer of 1 to 2000.

13. The method of claim 12, wherein the cells are one or more selected from the group consisting of erythrocytes, stem cells, and genetically engineered cells thereof.

14. The method of claim 12, wherein the liver disease is one or more selected from the group consisting of liver cancer, metastatic liver cancer, hepatic cirrhosis, hepatitis, and hepatic fibrosis.

15. (canceled)

16. A method of preparing a hyaluronic acid derivative comprising units represented by the following Chemical Formula 1 to 3 or a salt thereof, the method comprising:
a first step of preparing hyaluronic acid-aldehyde (HA-aldehyde) by reacting hyaluronic acid with an oxidant in the dark condition to open the ring structure of the hyaluronic acid; and
a second step of reacting the hyaluronic acid-aldehyde and the N-terminus of a water-soluble protein in an aqueous solution: 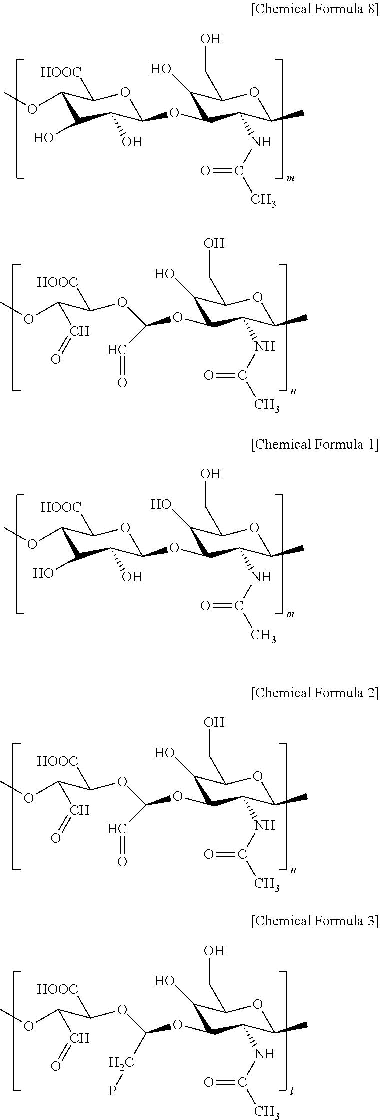
wherein P is a water-soluble protein; and
the sum of m, n and 1 is an integer of 50 to 10,000, the sum of n and 1 is an integer of 5 to 5,000, and 1 is an integer of 1 to 100.

17-24. (canceled)

25. The method of claim 16, further comprising a third step of blocking the remaining aldehyde groups which are unreacted with the protein, represented by the following Reaction Scheme 1: 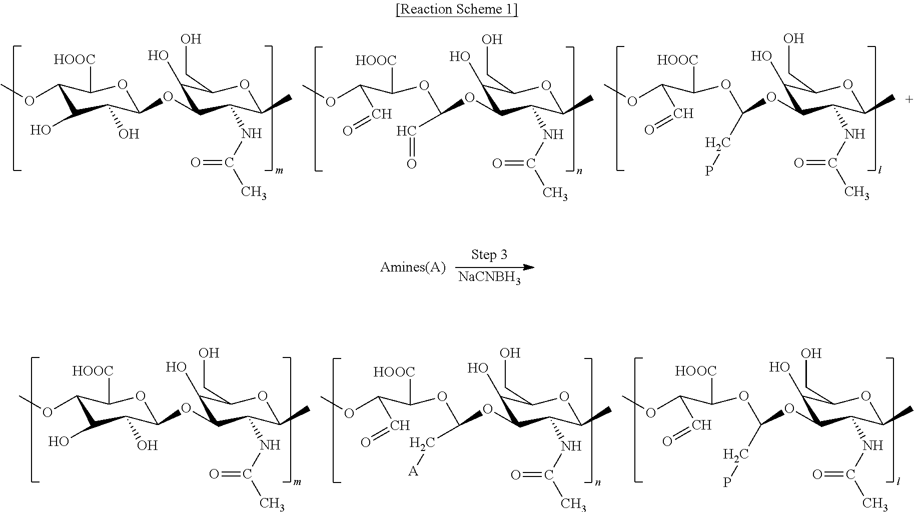
wherein P is a water-soluble protein;
the sum of m, n and 1 is an integer of 50 to 10,000, the sum of n and 1 is an integer of 5 to 5,000, and 1 is an integer of 1 to 100; and
A is an amine blocking material.

26-28. (canceled)


SEQUENCE LISTING (1 sequence)

SEQ ID NO: 1 - protein, 171 aa, Artificial Sequence
  Amino acid sequence of WGA protein
QRCGEQGSNMECPNNLCCSQYGYCGMGGDYCGKGCQDGACWTSKRCGSQAGGATCTNNQCCSQYGYCGFGAEYCGAGCQGGPCRADIKCGSQAGGKLCPNNLCCSQWGFCGLGSEFCGGGCQSGACSTDKPCGKDAGGRVCTNNYCCSKWGSCGIGPGYCGAGCQSGGCDG